(19) United States
(12) Patent Application Publication
(10) Pub. No.: US 2010/0274330 A1
(43) Pub. Date: Oct. 28, 2010

(54) DEVICE FOR TREATMENT OF BLOOD VESSELS USING LIGHT

(75) Inventors: Phillip Burwell, Port Townsend, WA (US); Zihong Guo, Bellevue, WA (US); Jennifer K. Matson, Portland, OR (US); Steven Ross Daly, Sammamish, WA (US); David B. Shine, Littleton, CO (US); Gary Lichttenegger, Woodinville, WA (US); Jean M. Bishop, Bothell, WA (US); Nick Yeo, Horsham (GB); Hugh Narciso, Santa Barbara, CA (US); James C. Chen, Clyde Hill, WA (US); William L. Barnard, Maple Valley, WA (US); Alexei N. Naimushin, Bellevue, WA (US)

Correspondence Address: LAW OFFICES OF RONALD M ANDERSON, 600 108TH AVE, NE, SUITE 507, BELLEVUE, WA, 98004, US

(73) Assignee: Light Sciences Oncology, Inc., Bellevue, WA (US)

(21) Appl. No.: 12/831,159

(22) Filed: Jul. 6, 2010

Related U.S. Application Data
(63) Continuation of application No. 12/101,069, filed on Apr. 10, 2008.
Application No. 12/101,069 is a continuation-in-part of application No. 10/888,572, filed on Jul. 9, 2004, now abandoned.
Application No. 10/888,572 is a continuation-in-part of application No. 10/799,357, filed on Mar. 12, 2004, now Pat. No. 7,252,677.
Application No. 10/799,357 is a continuation-in-part of application No. 11/834,572, filed on Aug. 6, 2007.
(60) Provisional application No. 60/486,178, filed on Jul. 9, 2003.
(60) Provisional application No. 60/455,069, filed on Mar. 14, 2003.

(51) Int. Cl. A61N 5/06

(52) U.S. Cl. 607/92

(57) ABSTRACT

Light generating devices for illuminating portions of vascular tissue to administer photodynamic therapy, and usable with, or including a distal protection device. A first device includes a hollow tip, a flushing lumen, a guidewire lumen, and at least one of a light source, and a hollow light transmissive shaft that is adapted to accommodate a light source. If desired, the device can include a balloon, so that a portion of a body lumen between the balloon and the distal protection device is isolated when the balloon is inflated. A second device includes inner and outer catheters, the outer catheter including a balloon, and the inner catheter including a light source encompassed by another balloon. Yet another device is a catheter having two balloons and a sleeve extending there between. Within the sleeve, the catheter includes a light source and an expanding member.

### RELATED APPLICATIONS

This application is a continuation of a copending patent application Ser. No. 12/101,069, filed on Apr. 10, 2008, which itself is a continuation-in-part of a patent application Ser. No. 10/888,572, filed on Jul. 9, 2004, which itself is based on a prior copending provisional application, Ser. No. 60/486,178, filed on Jul. 9, 2003, the benefit of the filing dates of which is hereby claimed under 35 U.S.C. §119(e) and 35 U.S.C. §120. patent application Ser. No. 10/888,572 is also a continuation-in-part of a prior copending application Ser. No. 10/799,357, filed on Mar. 12, 2004, which itself is based on a prior copending provisional application, Ser. No. 60/455,069, filed on Mar. 14, 2003, the benefit of the filing dates of which is hereby claimed under 35 U.S.C. §119(e) and 35 U.S.C. §120. This present application is further a continuation-in-part of a copending patent application Ser. No. 11/834,572, filed on Aug. 6, 2007, the benefit of the filing date of which is hereby claimed under 35 U.S.C. §120.

### BACKGROUND

Light activated drug therapy is a process whereby light of a specific wavelength or waveband is administered to tissue, to activate a light activatable drug that has been administered to the tissue. Significantly, only the drug actually exposed to light of the proper wavelength or waveband will be activated; thus, the process is highly selective and highly controllable, simply by controlling where the light is delivered. Light activatable drugs include those that when activated fluoresce, to facilitate a diagnostic function. The diagnostic function is particularly useful where the light activatable drug selectively accumulates at tissue of a certain type. Such selectivity can be enhanced by including a binding agent to the light activatable drug, where the binding agent selectively targets certain types of tissue, such as cancer cells. Light activatable drugs also include those that when activated can result in the destruction of adjacent cells/tissue. The term photodynamic therapy (PDT) is often employed where light of a specific wavelength or waveband is administered to tissue, to enable treatment of the tissue. The term photodynamic diagnosis (PDD) is often employed where light of a specific wavelength or waveband is administered to tissue, to enable a diagnosis of the tissue. In both diagnostic and therapeutic light activated drug therapy, the tissue is rendered photosensitive through the administration of a photoreactive or photosensitizing agent having a characteristic light absorption waveband. In therapeutic light activated drug therapy, the photoreactive agent is administered to a patient, typically by intravenous injection, oral administration, or by local delivery to the treatment site. Abnormal tissue in the body is known to selectively accumulate or retain (or otherwise absorb) certain photoreactive agents to a much greater extent than normal tissue. Once the abnormal tissue has taken up the photoreactive agent, the abnormal tissue can then be diagnosed or treated by administering light having one or more wavelengths or wavebands corresponding to the absorption wavelengths or wavebands of the photoreactive agent. Diagnostic light activated drug therapy reveals the location of the photoreactive agent, and hence, the location of the abnormal tissue, generally via a fluorescence signal, and therapeutic light activated drug therapy can be used to cause destruction (via necrosis or apoptosis) of the abnormal tissue.

Light activated drug therapy has proven to be very effective in destroying abnormal tissue, such as cancer cells, and has also been proposed for the treatment of vascular diseases, such as atherosclerosis and restenosis due to intimal hyperplasia. In the past, percutaneous transluminal coronary angioplasty (PTCA) has typically been performed to treat atherosclerotic obstruction of the coronary vasculature. Clinical results of PTCA have been enhanced by integrating one or more bare metal stents to prop open the revascularized vessel. However, restenosis due to vascular tissue proliferation at the site of the intervention often compromises the initial clinical benefit. A more recent treatment based on the use of drug eluting stents has reduced the rate of restenosis in coronary interventions. As effective as such therapies are in the coronaries, a new form of therapy is needed for treating peripheral arterial disease in the lower limbs, where the extent of disease and the challenges posed in this vascular bed are in many cases more demanding than in the coronary vasculature. New therapies are also required to treat more problematic coronary disease, such as unstable or vulnerable plaque, bifurcation disease, saphenous vein bypass graft disease, and diffuse long lesions (whether these occur in either the coronary or peripheral vasculatures).

As noted above, the objective of light activated drug intervention may be either diagnostic or therapeutic. In diagnostic applications, the wavelength of light is selected to cause the photoreactive agent to fluoresce, yielding information about the tissue without damaging the tissue. In therapeutic applications, the photonic energy within light of the characteristic wavelength/waveband delivered to the tissue is taken up by the photoreactive agent and, through inter-system crossing, this energy is transferred to molecular oxygen in the tissue within which the photoreactive agent is distributed. A highly reactive form of oxygen known as singlet oxygen is formed through this process. Singlet oxygen damages cellular and sub-cellular membranes, causing apoptotic or necrotic cell death, according to the quantities of drug and light in the tissue. The central strategy to inhibit arterial restenosis using light activated drug therapy, for example, is to cause a depletion of vascular smooth muscle cells, which are a source of neointimal cell proliferation. One of the advantages of light activated drug therapy is that it is a targeted technique, in that selective or preferential localization of the photoreactive agent to specific tissue enables only the selected tissue to be treated. Preferential localization of a photoreactive agent in areas of endovascular injury (for example, as caused by plaque debulking procedures such as angioplasty or atherectomy) or atherosclerotic disease, with little or no photoreactive agent being taken up by healthy or uninjured portions of the arterial wall, can therefore enable not only site-specific light activated drug therapy of an individual focal lesion, but also treatment of multifocal lesions within long vascular segments.

Light generating and delivery systems for light activated drug therapy are well known in the art. Delivery of light from a light source such as a laser, to the treatment site has typically been accomplished through the use of a single optical fiber delivery system with special light-diffusing tips. Exemplary prior art devices also include single optical fiber cylindrical diffusers, spherical diffusers, micro-lensing systems, an over-the-wire cylindrical diffusing multi-optical fiber catheter, and a light-diffusing optical fiber guidewire. Such prior art light activated drug therapy illumination systems generally employ remotely disposed high power lasers or solid state laser diode arrays, which are coupled to optical fibers for delivery of light to a treatment site. The disadvantages of using laser light sources include relatively high capital costs, relatively large size, significant inefficiencies in coupling light output from the laser and the optical fiber used to deliver light to the treatment site, complex operating procedures, and the safety issues that must be addressed when working with high power lasers. Accordingly, there is a substantial need for a light generating system that does not include a laser, and which generates light at the treatment site instead of at a remote point. For vascular applications of light activated drug therapy, it would be desirable to provide a light-generating apparatus having a minimal cross-section, a high degree of flexibility, and compatibility with a guidewire introduction system, so the light-generating apparatus can readily be delivered to the treatment site through a vascular lumen.

For vascular application of light activated drug therapy, it would further be desirable to provide a light-generating apparatus that is easily centered within a blood vessel, and which is configured to prevent light absorbent material, such as blood, from being disposed in the light path between the target tissue and the apparatus. Typically, an inflatable balloon catheter that matches the diameter of the blood vessel when the balloon is inflated is employed for centering apparatus within a vessel. Such devices also desirably occlude blood flow, enabling the light path to remain clear of obstructing blood.

Historically, the saphenous vein has been used to bypass stenotic coronary arteries during a PTCA surgical procedure. Increasing experience with postoperative follow-up of patients after saphenous vein bypass grafting has revealed a significant incidence of saphenous vein graft disease. Vein grafts develop endothelial proliferation as soon as they are placed in arterial circulation and after a few years, tend to develop atherosclerosis with thrombus formation. Vein graft atherosclerosis is often diffuse, concentric, and friable, with a poorly developed fibrous cap. Because of this characteristic, percutaneous interventions in saphenous vein grafts are limited by distal embolization, which can be extremely dangerous to a patient. Several types of catheter systems have been designed to capture atherothrombotic debris that embolize distally during vein graft intervention, where the intervention includes balloon dilation and/or stent placement. A distal protection device typically employs one of two approaches—a distal occlusion with a flow-occlusion balloon, followed by aspiration, and a distal occlusive filter. Neither approach is sufficient by itself. Therefore, it would be desirable to provide additional distal protection, to prevent accelerated vein graft disease, and to prevent distal embolization during interventions.

### SUMMARY

The present invention encompasses light generating devices for illuminating portions of vascular tissue to administer light activated drug therapy (PDT or PDD). Each embodiment includes one or more light sources adapted to be positioned inside a body cavity, a vascular system, or other body lumen. While the term “light source array” is frequently employed herein, because particularly preferred embodiments of this invention include multiple light sources arranged in a radial or linear array, it should be understood that a single light source can also be employed within the scope of this invention. Using a plurality of light sources generally enables larger treatment areas to be illuminated. Light emitting diodes (LEDs) are particularly preferred as light sources, although other types of light sources can be employed, as described in detail below. The light source that is used is selected based on the characteristics of a photoreactive agent with which the apparatus is intended to be used, since light of incorrect wavelengths or waveband will not cause the desired activation of the photoreactive agent and will therefore not generate singlet oxygen. An array of light sources can include light sources that provide more than one wavelength or produce light that covers one or more wavebands. Linear light source arrays are particularly useful to treat elongate portions of tissue within a lumen. Light source arrays used in this invention can also optionally include reflective elements to enhance the transmission of light in a preferred direction. Each embodiment described herein can beneficially include expandable members to occlude blood flow and to enable the apparatus to be centered in a blood vessel, even one that follows a tortuous path.

A key aspect of the light generating device of the present invention is that each embodiment is either adapted to be used with, or includes, a distal protection device. Interventions in vessels often results in distal embolization of atherosclerotic debris downstream, which can result in clinically significant events, including myocardial infarction, stroke, and renal failure. Distal protection devices trap or collect such debris in the blood, enabling its removal before unobstructed flow is restored. Studies relating to the use of distal protection devices indicate such devices reduce the incidence of major adverse cardiac events by as much as 50 percent.

The present invention uses at least one of an integrated light source element disposed on a distal end of an intra lumen device, and a substantially transparent hollow shaft disposed on a distal end of an intra lumen device, the hollow shaft being configured to accommodate a separate light source element. When a separate light source element is employed, the separate light source element is advanced through a working lumen in the intra lumen device and into the hollow shaft, after the intra lumen device is properly positioned in a body lumen. Preferably, the present invention also includes a hollow tip disposed distally of the light source element (or of the hollow shaft that is adapted to receive a separate light source element). The hollow tip includes an orifice at its distal end and an orifice on a side surface of the hollow tip, which enable the intra lumen device to be advanced over a guidewire, without the need to extend a guidewire lumen in the light source element (or in the hollow shaft into which a separate light source element will be introduced). A guidewire lumen is preferably included to enable the intra lumen device to be advanced over a guidewire; also preferably included is a flushing and aspiration lumen. The flushing and aspiration lumen enables a flushing fluid to be introduced into an isolated portion of a body lumen and enables the flushing fluid and any debris to be subsequently evacuated (i.e., aspirated) from the isolated portion of the body lumen.

In one embodiment of the present invention, a first intra lumen device does not include a distal protection device, but instead, is adapted to be used with existing distal protection devices. The first intra lumen device includes the light source element (or the hollow shaft adapted to accommodate a separate light source element), the hollow tip, the guidewire lumen, and the flushing lumen, all of which were discussed above. The first intra lumen device is adapted to be used with a guide catheter having an occlusion balloon at its distal end, and a distal protection device. A guidewire, distal protection device, and guide catheter are introduced into a body lumen, so that a distal end of the guidewire is disposed beyond the treatment area, the distal protection device is disposed distal of the treatment area, and a distal end of the guide catheter is disposed proximal of the treatment site. The first intra lumen device is advanced into the body lumen until the distal end of the first intra lumen device (including the light source element or the hollow shaft adapted to accommodate a light source element) is disposed adjacent to the treatment area, and between the distal end of the guide catheter and the distal protection device. If a separate light source element is used, the separate light source element is advanced into the hollow shaft adapted to accommodate the separate light source element. The distal protection device and the guide catheter balloon are activated, isolating the treatment area. Flushing fluid is introduced into the isolated area to displace blood that might interfere with light transmission, and the light source element is activated. Flushing fluid can be removed, along with any debris. Normal blood flow is allowed to resume for a period of time, and if required, additional light therapy is administered. Cycles of light therapy/diagnosis interspersed with periods of reperfusion can be used to reduce risk of ischemia in distal tissues caused by interruption in blood flow. The first intra lumen device can then be repositioned to treat other portions of the body lumen, if required. For example, in some cases, the light source element cannot illuminate the entire portion of the body lumen isolated by the guide catheter balloon and the distal treatment device, without being repositioned. A similar embodiment of the first intra lumen device includes a balloon disposed proximal of the light source element (or proximal of the hollow shaft adapted to accommodate a separate light source element), so that the guide catheter is not required to include a balloon.

Another embodiment of the present invention includes integrated distal protection devices. In one such embodiment, an outer guide catheter has an occlusion member (such as a balloon) disposed at its distal end, and an inner light emitting catheter. The light emitting catheter includes at least one of a light source element and a substantially transparent hollow shaft, and a hollow tip at its distal end (such that the light emitting catheter can be advanced over a guidewire without requiring a guidewire lumen to be included in the light element portion), as described above. The light emitting catheter further includes a generally light transmissive expandable member substantially encompassing the light source element (or the hollow shaft), so that the light source element can be centered within a body lumen, and so that the expandable member can displace blood that would otherwise block light from reaching the walls of the body lumen (and the target tissue) where the device is disposed. This embodiment further includes a distal protection device formed of a shape memory material that is disposed distal of the light source element. The distal protection device is activated by applying thermal energy to the shape memory material. Either a separate heating element is included, or the shape memory material overlays a portion of the light source element, so heat emitted by the light source element increases the temperature of the shape memory material, causing the distal protection device to deploy.

To use this embodiment of an intra lumen device, the guide catheter is positioned proximal of the treatment site, and the light emitting catheter is positioned so that the distal protection device is distal of the treatment site, and the light source element is disposed adjacent to the treatment site. The occlusion member is inflated, and the distal protection device is deployed, thus isolating a portion of the body lumen into which the device is deployed. The expandable member encompassing the light source element is expanded to perform angioplasty (if desired). Flushing fluid is introduced to remove debris, as discussed above. The expandable member is expanded once again, to displace blood that would interfere with light transmission, and the light source element is energized. Flushing fluid is introduced to remove any additional debris. Normal blood flow is allowed to resume for a period of time, and if required, additional light therapy is administered. Cycles of light therapy interspersed with periods of reperfusion can be used to reduce risk of ischemia in distal tissues caused by interruption in blood flow. The light emitting catheter can then be repositioned to treat other portions of the body lumen, if required.

Yet another embodiment of an intra lumen device that includes a distal protection device has a first and a second generally toroidal inflatable member (i.e., balloons) disposed at a distal end of the intra lumen device. An impermeable sleeve extends between the two inflatable members, forming a conduit within the sleeve through which blood (or other bodily fluid) is diverted when the inflatable members are inflated. Inflating the inflatable members results in a portion of a body lumen in which the device is disposed being isolated, without interrupting blood flow in the body lumen. The portion of the intra lumen device within the sleeve includes a light source element (or the hollow shaft adapted to accommodate a separate light source element). A light transmissive expandable member encompasses the light source element, as noted above. The intra lumen device includes a flushing lumen adapted to introduce (and remove) flushing fluid in the isolated portion of the body lumen (that portion between the inflatable members and the sleeve). It will be appreciated that the distal most inflatable member functions as a distal protection device. Preferably, the light source element is movable relative to the inflatable members, so that the light source element can be repositioned without deflating and re-inflating the inflatable members.

To use this intra lumen device, it is positioned within a body lumen so that a treatment area is disposed between the two inflatable members. The light source element is disposed adjacent the treatment site. The inflatable members are inflated, and the expandable member encompassing the light source element is expanded initially to perform angioplasty, if desired (note that the sleeve must be sufficiently flexible to accommodate this function). Flushing fluid is introduced to remove debris, as indicated above, and to keep the isolated portion free of blood that would interfere with light transmission. The expandable member is expanded once again, sufficiently to occlude blood flow within the sleeve (since the blood flow would interfere with light transmission), and the light source element is energized. Preferably, blood flow is occluded for less than about 50 seconds. Normal blood flow is allowed to resume for a period of time (preferably about 50 seconds), and if required, additional light therapy is administered. Cycles of light therapy interspersed with periods of reperfusion can be used to reduce risk of ischemia in distal tissues caused by interruption in blood flow. Flushing within the isolated portion is continued as needed to remove debris. The light source element can then be repositioned to treat other portions of the body lumen, as required.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A schematically illustrates a first embodiment of a light-generating device for use with a distal protection device during an intervention;

FIG. 1B schematically illustrates a guide catheter and a distal protection device being deployed in a vessel during an intervention;

FIG. 1C schematically illustrates the light-generating device of FIG. 1A, the guide catheter of FIG. 1B, and the distal protection device of FIG. 1B being used together during an intervention;

FIG. 1D is a cross-sectional view of the light-generating device of FIG. 1A;

FIG. 1E is a cross-sectional view of the light-generating device of FIG. 1A, showing the relative cross sectional footprints of the elongate flexible body, the light source array, the hollow tip, and the guidewire;

FIG. 2A schematically illustrates a second embodiment of a light-generating device for use with a distal protection device during an intervention;

FIG. 2B schematically illustrates a guide catheter and a distal protection device being deployed in a vessel during an intervention;

FIG. 2C schematically illustrates the light-generating device of FIG. 2A, the guide catheter of FIG. 2B, and the distal protection device of FIG. 2B being used together during an intervention;

FIG. 2D is a cross-sectional view of the light-generating device of FIG. 2A;

FIG. 3A schematically illustrates a heart, indicating the position of a saphenous vein graft;

FIG. 3B schematically illustrates a light-generating device with a distal protection device being used for an intervention;

FIGS. 3C and 3D are cross-sectional views of the light-generating device of FIG. 3B;

FIG. 3E schematically illustrates an embodiment of a light-generating device that is based on the light-generating device of FIG. 3B, in which heat from the light-generating device is used to deploy a shape memory material comprising the distal protection device;

FIG. 3F schematically illustrates a light-generating device based on the light-generating device of FIG. 3B, in which heat from a heating element is used to deploy the shape memory material comprising the distal protection device;

FIG. 3G is a cross-sectional view of an alternative guide catheter;

FIG. 3H is a cross-sectional view of an alternative guide catheter including a non occluding anchoring balloon;

FIG. 4A schematically illustrates another implementation of a light-generating device with a distal protection device, during an intervention;

FIG. 4B is a cross-sectional view of the light-generating device of FIG. 4A;

FIG. 4C is an enlarged view of a portion of FIG. 4A;

FIG. 5 schematically illustrates yet another embodiment of a light-generating apparatus suitable for intra vascular use in accord with the present invention;

FIG. 6 schematically illustrates a multicolor light array for use in the light-generating apparatus of FIG. 5;

FIGS. 7A and 7B schematically illustrate configurations of light arrays including strain relief features for enhanced flexibility for use in a light-generating apparatus in accord with the present invention;

FIG. 7C is cross-sectional view of a light-generating apparatus in accord with the present invention, showing one preferred configuration of how the light emitting array is positioned relative to the guidewire used to position the light-generating apparatus;

FIG. 7D schematically illustrates a portion of a light-generating apparatus in accord with the present invention, showing how in another preferred configuration, the light emitting array is positioned relative to the guidewire used to position the light-generating apparatus;

FIG. 8A schematically illustrates an embodiment of a light-generating apparatus in which light emitting elements are incorporated into a guidewire, as the apparatus is being positioned within a blood vessel;

FIG. 8B schematically illustrates another embodiment of a light-generating apparatus, in which light emitting elements are incorporated into a guidewire and which includes an inflatable balloon, showing the apparatus being positioned within a blood vessel;

FIG. 9A schematically illustrates a modified guidewire for use in the light-generating apparatus of FIGS. 8A and 8B;

FIGS. 9B-9D are cross-sectional views of the guidewire of FIG. 9A, showing details of how the light emitting elements are integrated into the guidewire;

FIG. 10A schematically illustrates still another embodiment of a light-generating apparatus, which includes a plurality of inflatable balloons, as the apparatus is being positioned within a blood vessel;

FIG. 10B is a cross-sectional view of the light-generating apparatus of FIG. 10A;

FIG. 11A schematically illustrates an alternative configuration of a light-generating apparatus including a plurality of inflatable balloons, as the apparatus is being positioned within a blood vessel;

FIG. 11B is a cross-sectional view of the light-generating apparatus of FIG. 11A; and

FIG. 12 schematically illustrates a plurality of balloons included with a light-generating apparatus in accord with the present invention.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

Unless otherwise defined, it should be understood that each technical and scientific term used herein and in the claims that follow is intended to be interpreted in a manner consistent with the meaning of that term as it would be understood by one of skill in the art to which this invention pertains. The drawings and disclosure of all patents and publications referred to herein are hereby specifically incorporated herein by reference. In the event that more than one definition is provided herein, the explicitly defined definition controls.

Various embodiments of light-generating devices that either incorporate distal protection devices, or are adapted to be used with a distal protection device, are described herein. An objective of administering light activated drug therapy with the present invention may be either diagnostic (i.e., PDD), wherein the wavelength or waveband of the light being produced is selected to cause the photoreactive agent to fluoresce, thus yielding information about a target tissue, or therapeutic (i.e., PDT), wherein the wavelength or waveband of the light delivered to photosensitized tissue under treatment causes the photoreactive agent to undergo a photochemical interaction with molecular oxygen in the tissue yielding highly reactive singlet oxygen, causing biological changes in the tissue within which the photoreactive agent is distributed.

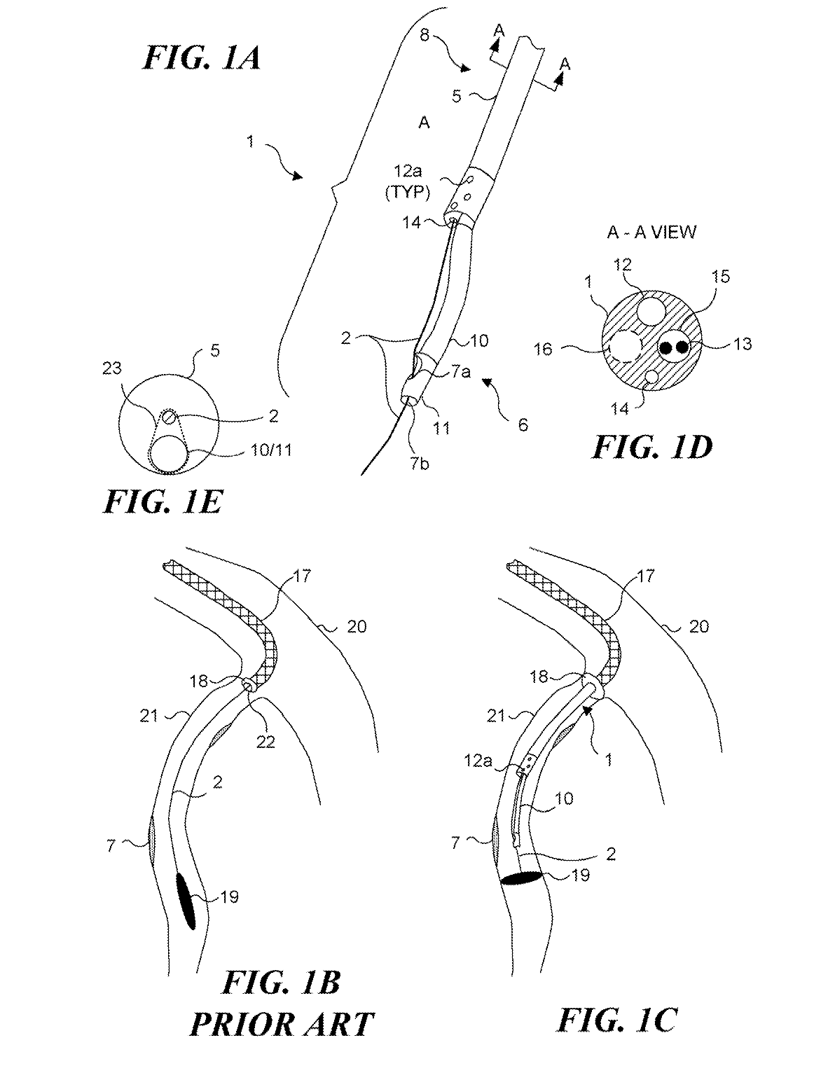

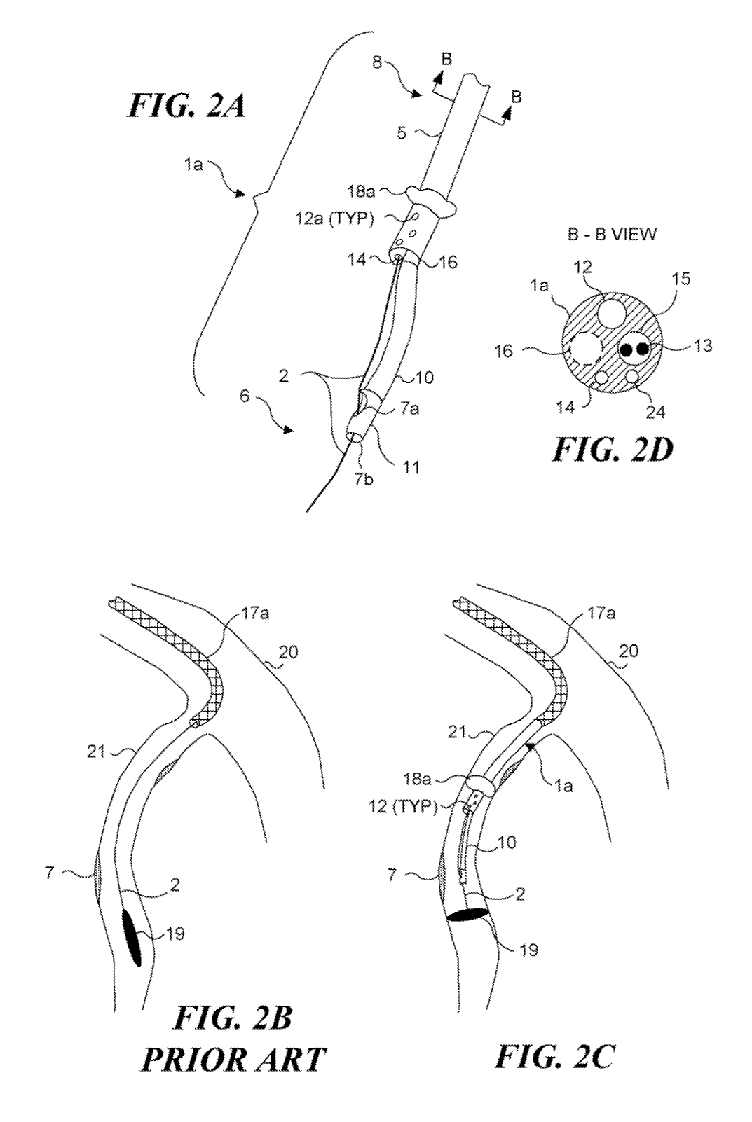

Referring to FIG. 1A, a light-generating device 1 comprises a multi-lumen catheter having an elongate flexible body 5, formed from a suitable biocompatible material, such as a polymer or metal. Light generating device 1 is adapted to be used with prior art distal protection devices, as explained in greater detail below, and includes a distal end 6, a proximal end 8 (normally disposed outside a body lumen and configured to enable light-generating device 1 to be manipulated), a flushing lumen 12, a guidewire lumen 14, an optional working lumen 16, an optional power lumen 15, and a light source array 10 (see FIG. 1D, which shows lumens 12, 14, 15, and 16). Generally, either a working lumen or a power lumen will be included, as discussed in detail below. The relative configuration of the lumens as shown in FIG. 1D is intended to be exemplary, and other configurations can be employed in the alternative. Thus, the relative orientations of the lumens of FIG. 1D is not intended to be limiting of the present invention. Furthermore, the lumens shown in FIG. 1D are not drawn to scale, and the relative sizes of the lumens shown are exemplary, rather than controlling. These comments, which specifically pertain to the cross sectional view of FIG. 1D, also apply to the cross sectional views of FIGS. 2D, 3C, 3D, and 4B.

Guidewire lumen 14 enables elongate flexible body 5 to be advanced over a guidewire, and flushing lumen 12 enables a flushing fluid to be introduced into a body lumen proximate distal end 6 of elongate flexible body 5. Guidewire lumen 14 comprises a hollow conduit of a diameter sufficient to accommodate a guidewire therein and extends between distal end 6 and proximal end 8. As indicated in FIG. 1A, the guidewire is disposed externally of light-generating device 1 near a light source array 10, so that light source array 10 is not required to include a guidewire lumen. Flushing lumen 12 is preferably used to convey saline, or another appropriate fluid (such as a light scattering medium such as Intralipid, or an optically clear, biocompatible fluid, or a radio-opaque medium, or a mixture of one of more of these), to displace bodily fluids (such as blood) proximate distal end 6, when light-generating device 1 is disposed in a body lumen. Such bodily fluids (especially blood) may undesirably interfere with the transmission of light from light source array 10 to target tissue. The flushing fluid is introduced into the body lumen via ports 12a that are disposed on distal end 6 of elongate flexible body 5, proximate light source array 10.

Light source array 10 includes one or more LEDs coupled to conductive traces (not shown) that are electrically connected to conductors 13 extending proximally through a power lumen 15 of light-generating device 1, to an external power supply and control device (not shown). Thus, conductors 13 enable the LEDs to be coupled to a power source. As an alternative to LEDs, other sources of light maybe used, such as organic LEDs, superluminescent diodes, laser diodes, fluorescent light sources, incandescent sources, and light emitting polymers. Light source array 10 is preferably encapsulated in silicone, or another biocompatible polymer, and is coupled to the distal end of elongate flexible body 5.

Optional working lumen 16 is configured to enable a non integrated light source array to be employed. Instead of including integrated light source array 10, light-generating device 1 can be configured without any integrated light source, so that a separate light source array is advanced to the target area through the working lumen after light-generating device 1 is properly positioned in the body lumen. Of course, if a non integral light source array is used, power lumen 15 is not necessary (the power leads for the separate light source element being disposed in the working lumen) and may thus be omitted. If a separate light source array is used, then a hollow, light transmissive shaft is disposed between a tip 11 and ports 12a (i.e., if a separate light source array is employed, then reference numeral 10 corresponds to a hollow, light transmissive shaft configured to accommodate a light source array).

Distal end 6 of light-generating device 1 includes hollow tip 11 coupled to a distal end of light source array 10, (or to the hollow shaft if used in place of light source array 10), with an outwardly facing orifice 7a, as well as a distal orifice 7b, which enable light-generating device 1 to be advanced over a guidewire 2. Note that guidewire lumen 14 does not extend into light source array 10, and thus, the guidewire is disposed external of and proximate to light source array 10, such that the portion of the guidewire proximate to the light source array is exposed to the body lumen, as indicated in FIG. 1C. To position light-generating device 1 in a body lumen, a guidewire 2 is introduced into an artery (or other body lumen) and advanced until the guidewire is disposed adjacent a treatment area (generally an arterial lesion). Elongate flexible body 5 is then advanced over guidewire 2, until distal end 6 is adjacent to the treatment area.

FIG. 1B schematically illustrates a prior art distal protection device 19, such as a PERCUSURGE™ or an ANGIOGUARD™ Filter, that has been advanced through a guide catheter 17, through aorta 20, for placement at an anastomosis of a saphenous vein graft 21. Guide catheter 17 includes an occlusion balloon 18 disposed near a distal end 22 of guide catheter 17. As shown in FIG. 1C, light generating device 1 is advanced into saphenous vein graft 21, until it is disposed between balloon 18 and distal protection device 19. The guide catheter includes a working lumen that is larger than light-generating device 1, so that light-generating device 1 is advanced to the treatment site within the working lumen of the guide catheter.

Once light-generating device 1 is properly positioned, occlusion balloon 18 is inflated to block blood flow. Saline solution (or an another biocompatible solution that facilitates light transmission) is flushed through flushing lumen 12 of light generating device 1 to displace the blood in saphenous vein graft 21, thereby facilitating light illumination of target tissue 7. Distal protection device 19 is activated (i.e., expanded), and light-generating device 1 is energized to illuminate target tissue 7. Target tissue 7 will preferably have previously been treated with a photoreactive agent, but if the particular photoreactive agent employed is rapidly taken up by target tissue 7, light generating device 1 can be used to deliver the photoreactive agent through flushing lumen 12, or through a dedicated drug delivery lumen (not shown).

Distal protection device 19 is used to capture atherosclerotic debris that may be generated during the treatment of target tissue 7. Such debris, if allowed to escape downstream, may result in clinically significant and undesirable events, including myocardial infarction, stroke, and renal failure. As noted above, studies have shown that the use of distal protection devices reduces the incidence of major adverse cardiac events by as much as 50 percent. Light-generating device 1 can be moved within saphenous vein graft 21 to enable the light source array to illuminate other target tissue, if the target area extends beyond an area that can be illuminated at one time.

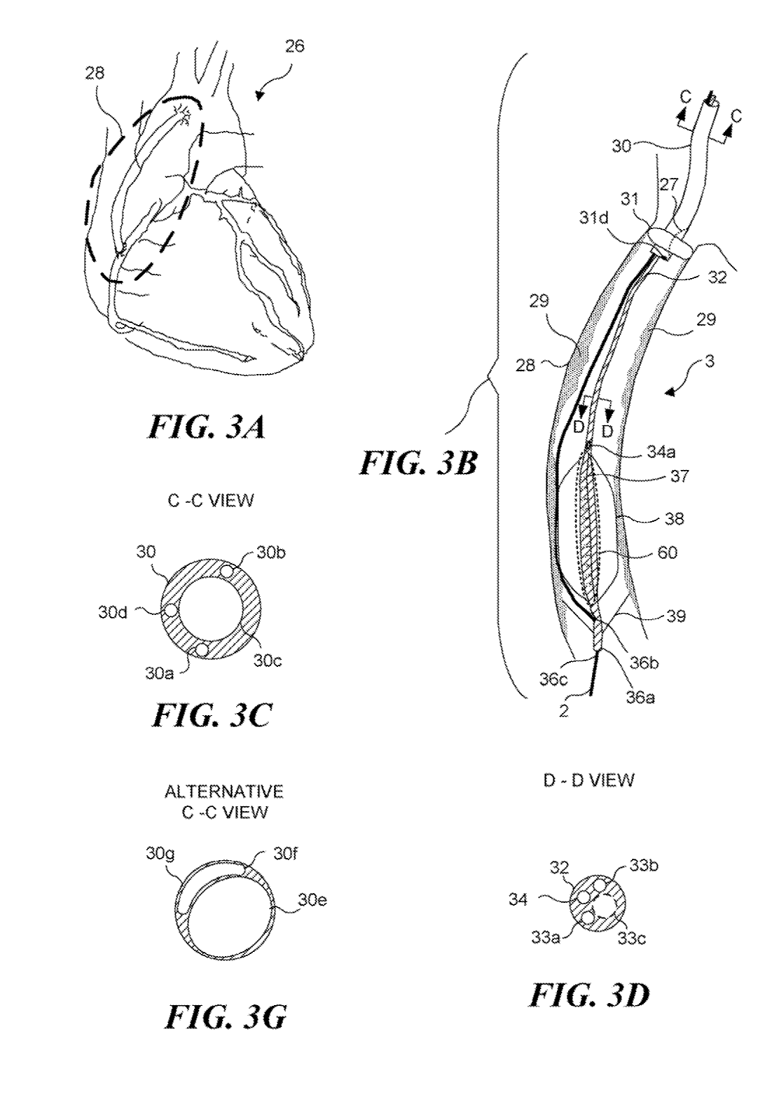

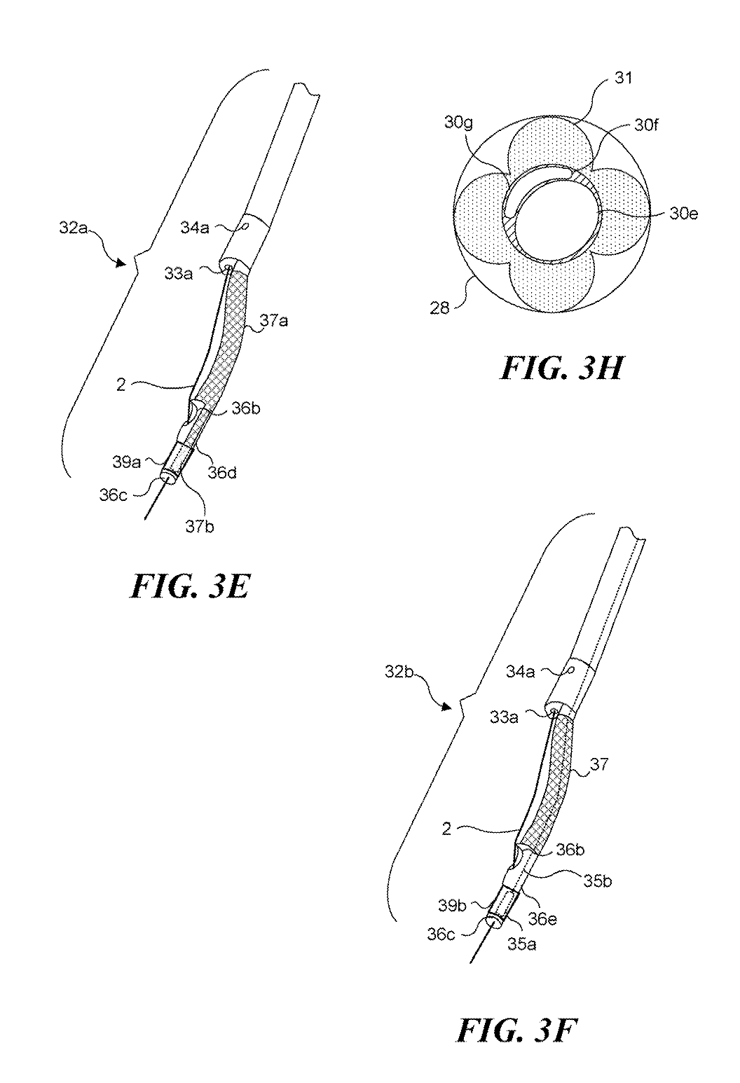

It should be noted that the light source array 10 and hollow tip 11 each have a diameter smaller than that of elongate flexible body 5. Note that as the guidewire extends beyond elongate flexible body 5 to hollow tip 11, the guidewire is parallel to and external of the light source array. The reduced diameter of the light source means that the guide wire does not radially extend into the body lumen beyond elongate flexible body 5. In other words, a cross sectional footprint of the guidewire, the hollow tip, and the light source array are smaller than a cross sectional footprint of elongate flexible body 5. The structures of FIGS. 2A, 3E, and 3F (discussed in detail below) share this characteristic. This concept is schematically illustrated in FIG. 1E, which is an end view of the apparatus of FIG. 1A, showing the cross sectional foot prints of elongate flexible body 5, guidewire 2, light source array 10 and hollow tip 11 (note that the light source array and the hollow tip are shown as overlapping; in general, each will have approximately the same diameter, however, even if their diameters differ, each will still have a diameter that is reduced as compared to the elongate flexible body). Note that combined cross section footprint 23 for the guidewire, the hollow tip, and the light source array is smaller than the footprint of the elongate flexible body.

FIGS. 2A-2D schematically illustrate a related embodiment of a light generating device 1a, which is intended to be used in a fashion similar to that described above, in regard to light-generating device 1. The difference between light-generating device 1 (FIGS. 1A, 1C, and 1D) and light-generating device 1a (FIGS. 2A, 2C, and 2D) is that light-generating device 1a includes a low-pressure compliant occlusion balloon 18a, and an inflation lumen 24 (see FIG. 2D). Accordingly, a guide catheter 17a (see FIGS. 2B and 2C) is not required to include an occlusion balloon, as is necessary for guide catheter 17 of FIGS. 1B and 1C. Because the guide catheter is not required to include a balloon, it is possible, but less preferred, for the guide catheter to be smaller than optional working lumen 16 of light-generating device 1a, so that light-generating device 1a is advanced to the treatment site over the guide catheter.

FIG. 3A schematically illustrates a heart 26, generally indicating the position of a saphenous vein graft 28, a portion of which is depicted in greater detail in FIG. 3B. The portion of saphenous vein graft 28 shown in FIG. 3B includes treatment areas 29 (typically having lesions or plaque). Yet another embodiment of a light-generating device is shown in FIG. 3B. Note that while light-generating device 1 of FIGS. 1A-1D, and light-generating device 1a of FIGS. 2A-2D each are intended to be used with a prior art distal protection device, the light generating devices discussed in connection with FIGS. 3A-3G include a distal protection member. Referring to FIG. 3B, a light-generating device 3 includes a guide catheter 30 and a multi-lumen light-generating catheter 32. Guide catheter 30 includes a low pressure occlusion balloon 31 (disposed near the distal end of guide catheter 30). Also, as shown in FIG. 3C, guide catheter 30 has a guidewire lumen 30a, an inflation lumen 30b (adapted to enable low pressure occlusion balloon 31 to be selectively inflated), a working lumen 30c, and an aspiration lumen 30d. Working lumen 30c is configured to accommodate light-generating catheter 32, so that light-generating catheter 32 can be advanced to a treatment site within the working lumen of guide catheter 30. Aspiration lumen 30d enables a flushing fluid introduced via light-generating catheter 32 (described in detail below) to be removed from the body lumen. However, if desired, the flushing lumen in light-generating catheter 32 can be used both to introduce a flushing fluid, and to aspirate the flushing fluid previously introduced, so that aspiration lumen 30d is then not required.

FIG. 3G schematically illustrates an alternative guide catheter 30g, which includes only two lumens. Some practitioners prefer a large working lumen over a plurality of smaller lumens. Guide catheter 30g includes a relatively large working lumen 30e (which combines the functions of guidewire lumen 30a, working lumen 30c, and aspiration lumen 30d of guide catheter 30) and an inflation lumen 30f. As noted above, the inflation lumen is used to selectively inflate low pressure occlusion balloon 31.

Light-generating catheter 32 has an elongate flexible body formed from a suitable biocompatible material, such as a polymer or metal. As shown in FIG. 3D, light-generating catheter 32 also has a plurality of lumens, including a flushing lumen 34, a power lumen 33a, an inflation lumen 33b, and an optional working lumen 33c. It should be recognized that such a lumen configuration is intended to be exemplary, rather than limiting.

Referring back to FIG. 3B, a flushing medium is introduced into a body lumen into which light-generating catheter 32 is disposed through one or more ports 34a. Once again, the flushing medium may be saline solution or any other appropriate medium that is suitable to displace the bodily fluids (such as blood) in a body lumen, to facilitate light illumination of the target tissue. Power lumen 33a is a hollow conduit of a diameter sufficient to accommodate electrical conductors therein, and extends between a distal end of light-generating catheter 32 and a proximal end of light-generating catheter 32, thus enabling the light sources (discussed below) to be electrically coupled to a power source. Note that FIG. 1D shows leads 13 that similarly couple light sources disposed proximate a distal end of a device to an external power source. It should be recognized that the terms electrical conductors and lead can be used interchangeably, and includes structures including but not limited to metallic conductors, wires, and flexible circuits. As indicated in FIG. 3B, the guidewire is disposed externally of light-generating catheter 32 near a light emitting portion, so that the light emitting portion is not required to include a guidewire lumen. In other words, a portion of guidewire 2 proximate expandable member 38 (and light source array 37, which is disposed within the expandable member) is not enclosed in a guidewire lumen, and is thus exposed to the body lumen, generally as discussed above with respect to FIG. 1A. As shown in FIG. 3B, an outer surface of expandable member 38 deflects that portion of guidewire 2 toward the wall of the body lumen. The distal end of light-generating catheter 32 includes a hollow tip 36a with an orifice 36b that faces toward a wall of the body lumen, and a distal orifice 36c (in a configuration similar to that shown in FIG. 1A for light-generating device 1). Orifices 36b and 36c facilitate the advancement of light-generating catheter 32 over guidewire 2.

Note that light-generating catheter 32 does not require a guide-wire lumen, as light-generating catheter 32 is advanced through the working lumen of guide catheter 30. If desired, light-generating catheter 32 can incorporate a guide-wire lumen, to enable light-generating catheter 32 to be used independent of a guide catheter.

Light-generating catheter 32 includes a light source array 37, which can optionally be coupled to collection optics (not shown). As discussed above in connection with light source array 10 of FIG. 1A, light source array 37 may include one or more LEDs coupled to conductive traces that are electrically connected to leads extending proximally through a lumen of the light generating catheter to an external power supply and control device (not shown). As an alternative to LEDs, other sources of light may be used, such as organic LEDs, superluminescent diodes, laser diodes, fluorescent light sources, incandescent sources, and light emitting polymers. Light source array 37 is preferably encapsulated or otherwise covered with a substantially optically transparent (at least with regard to the wavelengths emitted by light source array 37) biocompatible polymer, such as silicone. Light source array 37 can be integral to light-generating catheter 32 (in which case, light-generating catheter 32 preferably includes a power lumen to convey the electrical leads that are employed to couple the light source array to an external power supply), or light source array 37 can be a separate component that is advanced to the treatment site using optional working lumen 33c, after light-generating catheter 32 is properly positioned in the body lumen. If light source array 37 is a separate component, then light-generating catheter 32 includes a transparent hollow shaft 60, adapted to accommodate light source array 37 (such a shaft is also described above, in connection with FIG. 1A and light-generating device 1). Note that if the light source array 37 is a separate component, a power lumen is not required, and the light source array 37 (and any electrical conductors it requires) can be advanced through the optional working lumen. As will be discussed in detail below, the concepts disclosed herein include a light emitting guidewire that can be used to implement light source array 37 as a separate component.

Light-generating catheter 32 also includes an expandable member 38, for centering the distal end of light-generating catheter 32, and for either occluding blood flow or for performing angioplasty (or both). Inflation lumen 33b is adapted to selectively control the inflation of expandable member 38, which is preferably secured to the distal portion of light-generating catheter 32 so as to encompass light source array 37. Expandable member 38 comprises a suitable biocompatible material, such as, polyurethane, polyethylene, fluorinated ethylene propylene (FEP), polytetrafluoroethylene (PTFE) or PET (polyethylene terephthalate), and preferably, is substantially light transmissive, since light from light source array 37 must freely pass through expandable member 38 to reach the target tissue. Distal of expandable member 38 and orifice 36b is a shape memory filter 39 that traps and removes emboli and/or other debris from the body lumen within which light-generating catheter 32 is being used.

Shape memory filter 39 moves between its first and second positions in response to a temperature change, preferably, an increase in temperature. An application of heat increases the temperature of the shape memory material above its transition temperature. The shape memory material memorizes a certain shape at a certain temperature and can be selectively activated to return to its memorized shape by applying heat to the shape memory material so that it is heated above the transition temperature. Preferably the shape memory material is a polymer; such shape memory materials are well known in the art and need not be described herein in detail. The first position of shape memory filter 39 corresponds to an un-deployed configuration, wherein shape memory filter 39 generally conforms to the distal end of the light-generating catheter 32. The second position of shape memory filter 39 corresponds to a deployed configuration, wherein shape memory filter 39 generally expands outwardly and away from light-generating catheter 32, until the shape memory filter contacts the walls of the body lumen in which light-generating catheter 32 is deployed, thereby preventing debris from moving past shape memory filter 39.

When light-generating device 3 is in use, guide catheter 30 is introduced into a body lumen and positioned proximal of a treatment area. Then, light-generating catheter 32 is advanced through guide catheter 30 (and over guidewire 2, distal of guide catheter 30) until light source array 37 is disposed adjacent the treatment area. While the light-generating catheter 32 is being advanced over the guidewire to a treatment site, shape memory filter 39 is not deployed. When light-generating catheter 32 is positioned adjacent to the treatment site, shape memory filter 39 is deployed into its second position. Occlusion balloon 31 is inflated, and expandable member 38 is inflated and deflated to perform angioplasty (if desired).

Saline solution is then introduced to the isolated portion of the body lumen (i.e., to the portion between occlusion balloon 31 and shape memory filter 39) via flushing lumen 34 and removed via aspiration lumen 30d. As noted above, flushing and aspiration could be carried out using a single lumen, by first flushing and then aspirating through the lumen. The use of a separate flushing lumen and a separate aspiration lumen enable a circulating flow to be achieved, so that more debris can be removed in a shorter time. Flushing not only removes debris, which might get past shape memory filter 39 as light-generating catheter 32 is removed, but also maintains a clear light transmission path to the body lumen wall, keeping the portion of the body lumen between balloon 31 and shape memory filter 39 essentially free of blood and debris. Expandable member 38 is then again inflated to facilitate the transmission of light from light source array 37 to the body lumen wall. Preferably, light source array 37 is rotated within catheter 32, to enable all portions of the lumen walls around the light source array to be illuminated. Alternatively, the light source array can include light sources disposed so that light is emitted outwardly of the light source array through substantially a full 360 degrees of arc.

As noted above, shape memory filter 39 is preferably deployed by using heat. FIGS. 3E and 3F schematically illustrate different embodiments for applying the required thermal energy to shape memory filter 39. Each of FIGS. 3E and 3F includes a light generating catheter substantially similar to light-generating catheter 32, except for the modification discussed in detail below to enable shape memory filter 39 to be heated by the light source. In each of FIGS. 3E and 3F, expandable member 38 has been omitted, to reduce the complexity of those Figures.

FIG. 3E schematically illustrates a light-generating catheter 32a (with the expanding member not shown, as noted above). A light source array 37a extends into a hollow tip 36d. A shape memory filter 39a is disposed distal of orifice 36b, to ensure that the guidewire does not interfere with the filter when it is in the deployed position. In FIG. 3E, shape memory filter 39a is not yet deployed, and part of shape memory filter 39a overlays a portion 37b of light source array 37a. Energizing light source array 37a produces heat that is absorbed by shape memory filter 39a, causing the filter to deploy.

FIG. 3F illustrates a related embodiment, in which a heater, rather than the light-generating array, is used to provide the heat that changes the temperature of the shape memory material comprising the filter (the distal protection device). In FIG. 3F, a light-generating catheter 32b is shown. A hollow tip 36e includes orifice 36b, orifice 36c, and a heating element 35a. A shape memory filter 39b is disposed distal of orifice 36b, again to ensure that the guidewire does not interfere with the filter when it is in the deployed position. Shape memory filter 39b does not overlie light source array 37 in light-generating catheter 32b. Instead, shape memory filter 39b is disposed adjacent to heating element 35a, so that the heat produced by energizing heating element 35a causes shape memory filter 39b to deploy. Electrical lead 35b couples heating element 35a to an external power source (not shown). Preferably, heating element 35a is a resistive heating element, such as a nichrome wire, although other types of heating elements can alternatively be employed.

It should be recognized that if desired the distal protection device can be eliminated from the light generating catheters described above.

Referring once again to FIG. 3B, a unique aspect of light-generating catheter 32 is that it is independently positionable relative to the guide catheter. In use, the guide catheter is initially positioned (and locked in place by inflating balloon 31), and then light-generating catheter 32 is advanced beyond the distal end of the guide catheter to treat a portion of the blood vessel distal of the guide catheter. Note in FIG. 3B the lesions (treatment areas 29) are much longer than the light source array in light-generating catheter 32, thus, the light-generating catheter will need to be repositioned several times in order to treat the entire lesion (recognizing that the treatment area being treated is not limited to lesions, and generally includes longitudinal segments of the blood vessel). This can be achieved by keeping balloon 31 inflated (such that the guide catheter remains fixed in position), and moving the light-generating catheter relative to the distal end of the guide catheter, in order to treat other portions of the vessel.

Particularly if the treatment area is large enough to require light-generating catheter 32 to be repositioned several times, it will be beneficial to provide a bypass for blood flow occluded by balloon 31. Such a bypass can be implemented by adding a bypass lumen 27 to the distal end of the guide catheter. Such a bypass can also be implemented by configuring balloon 31 to leak, in the sense balloon 31 is configured to ensure that the guide catheter remains fixed in position, without fully occluding blood flow distal of the balloon. In at least one embodiment (see FIG. 3H), balloon 31 has a cross sectional shape when inflated such that balloon 31 does not engage a full 360 degrees of the blood vessel wall. The number of “lobes” of the balloon engaging the vessel wall is not critical, so long as balloon 31 can prevent the guide catheter from moving out of position when the balloon is inflated. Rather than employing a single balloon with a plurality of radial lobes, a plurality of individual balloons could be employed to perform the same function.

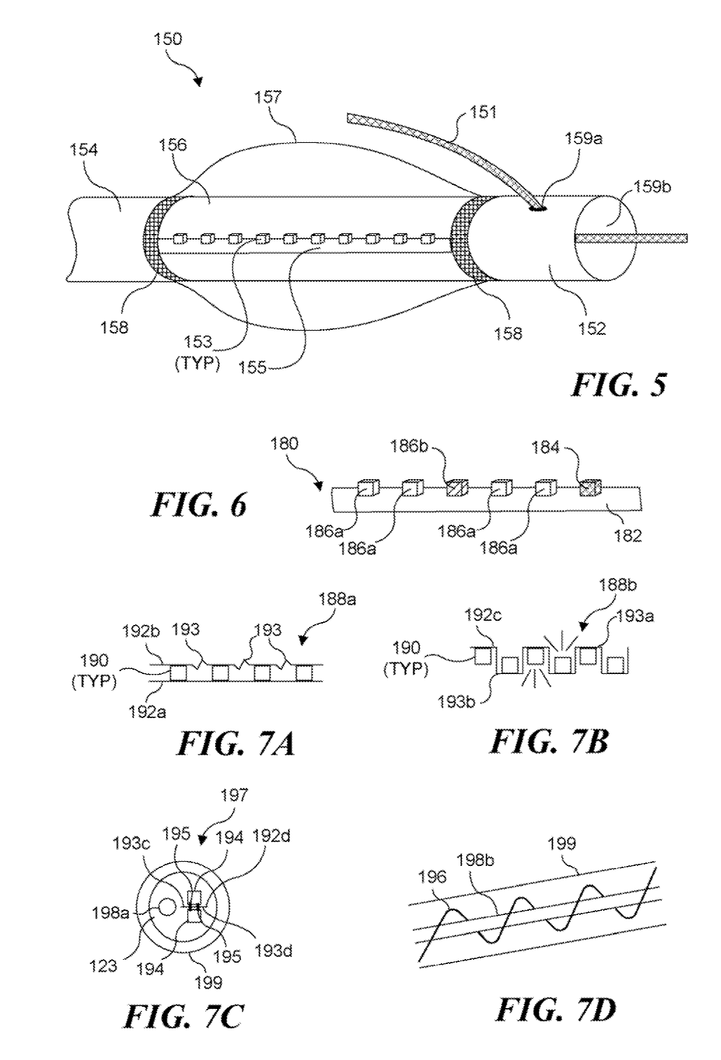

Another useful modification to the guide catheter and light generating catheter of FIG. 3B is the incorporation of reference markers, such as radio-opaque markers (described in greater detail below in connection with the description of FIG. 5). Such markers include metallic elements integrated into portions of a catheter to enable the position of the catheter to be accurately determined. Such markers also include an easily imaged fluid that can be used to inflate balloons. It will be most beneficial to incorporate such markers in the light-generating catheter, such that the locations of portions of the vessel being treated can be logged, so that portions of the vessel do not receive multiple treatments unless multiple treatments are desired. It will be even more beneficial to incorporate such markers both in the light-generating catheter and in the guide catheter so that precise location of successive light treatments can be registered with respect to the fixed tip of the guide catheter, so as to avoid either gaps or overlaps between PDT treatment stations as the light-generating catheter is moved along the vessel being treated. It may be less useful to include such markers in the guide catheter alone, although such an embodiment is encompassed by the disclosure herein.

Yet another modification to the guide catheter and light generating catheter of FIG. 3B is to utilize a removable light source array, rather than a fixed array. In such a case, the light emitting catheter will desirably include a working lumen to accommodate the removable light source array (such working lumens have already been described and illustrated elsewhere in this disclosure). In such an embodiment, the side facing distal tip is not required, as the working lumen can accommodate a guidewire. Guidewires including integral light sources are disclosed below. Thus, such an embodiment can incorporate a combination guide wire/light source array, or a lumen that at one time accommodates a guidewire and then later accommodates a removable light source array (after the guidewire is withdrawn).

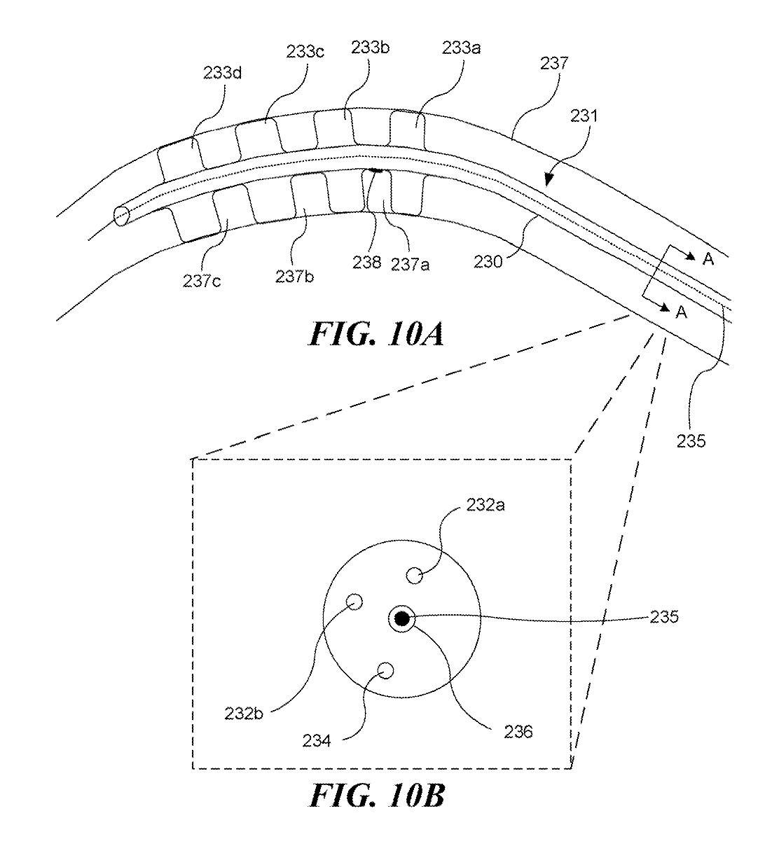

Still another modification to the guide catheter and light-generating catheter of FIG. 3B is to utilize a multi-lobed balloon to cover the light source array, or a plurality of smaller individual balloons. Such structures are discussed in greater detail in connection with FIGS. 10A, 11A, and 12.

FIG. 5 schematically illustrates yet another embodiment of a light-generating catheter, which is similar to light-generating catheters 32, 32a, and 32b, but which does not necessarily include the distal protection device (though it should be recognized that such an embodiment is encompassed within the scope of the present disclosure). Thus, FIG. 5 illustrates a light-generating apparatus 150. As with the embodiments described above (i.e., light-generating catheters 32, 32a, and 32b), light-generating apparatus 150 is preferably based on a multi-lumen catheter and includes an elongate, flexible body formed from a suitable biocompatible polymer or metal, which includes a distal portion 152 and a proximal portion 154. A plurality of light emitting devices 153 are disposed on a flexible, conductive substrate 155 encapsulated in a flexible cover 156 (formed of silicone or other flexible and light transmissive material). Light emitting devices 153 and conductive substrate 155 together comprise a light source array. Preferably, light emitting devices 153 are LEDs, although other light emitting devices, such as organic LEDs, super luminescent diodes, laser diodes, or light emitting polymers can be employed. Each light emitting device 153 preferably ranges from about 1 cm to about 10 cm in length, with a diameter that ranges from about 0.5 mm to about 5 mm. Flexible cover 156 can be optically transparent or can include embedded light scattering elements (such as titanium dioxide particles) to improve the uniformity of the light emitted from light-generating apparatus 150. While not specifically shown, it should be understood that proximal portion 154 includes electrical conductors enabling conductive substrate 155 to be coupled to an external power supply and control unit, as described above for the embodiments that have already been discussed.

The array formed of light emitting devices 153 and conductive substrate 155 is disposed between proximal portion 154 and distal portion 152, with each end of the array being identifiable by radio-opaque markers 158 (one radio-opaque marker 158 being included on distal portion 152, and one radio-opaque marker 158 being included on proximal portion 154). Radio-opaque markers 158 comprise metallic rings of gold or platinum. Light-generating apparatus 150 includes an expandable member 157 (such as a balloon) preferably configured to encompass the portion of light-generating apparatus 150 disposed between radio-opaque markers 158 (i.e., substantially the entire array of light emitting devices 153 and conductive substrate 155). As discussed above, expandable member 157 enables occlusion of blood flow past distal portion 152 and centers the light-generating apparatus. Where an expandable member is implemented as a fluid filled balloon, the fluid acts as a heat sink to reduce a temperature build-up caused by light emitting devices 153. This cooling effect can be enhanced if light-generating apparatus 150 is configured to circulate the fluid through the balloon, so that heated fluid is continually (or periodically) replaced with cooler fluid. Preferably, expandable member 157 ranges in size (when expanded) from about 2 mm to 10 mm in diameter. Preferably such expandable members are less than 2 mm in diameter when collapsed, to enable the apparatus to be used in a coronary vessel. Those of ordinary skill will recognize that catheters including an inflation lumen in fluid communication with an inflatable balloon, to enable the balloon to be inflated after the catheter has been inserted into a body cavity or blood vessel, are well known. While not separately shown, it will therefore be understood that light-generating apparatus 150 (particularly proximal portion 154) includes an inflation lumen. When light emitting devices 153 are energized to provide illumination, expandable member 157 can be inflated using a radio-opaque fluid, such as Renocal 76™ or normal saline, which assists in visualizing the light-generating portion of light-generating apparatus 150 during computerized tomography (CT) or angiography. The fluid employed for inflating expandable member 157 can be beneficially mixed with light scattering material, such as Intralipid, a commercially available fat emulsion, to further improve dispersion and light uniformity.

Light-generating apparatus 150 is positioned at a treatment site using a guidewire 151 that does not pass through the portion of light-generating apparatus 150 that includes the light emitting devices. Instead, guidewire 151 is disposed external to light-generating apparatus 150—at least between proximal portion 154 and distal portion 152. Thus, the part of guidewire 151 that is proximate light emitting devices 153 is not encompassed by expandable member 157. Distal portion 152 includes an orifice 159a, and an orifice 159b. Guidewire 151 enters orifice 159a, and exits distal portion 152 through orifice 159b. It should be understood that guidewire 151 can be disposed externally to proximal portion 154, or alternatively, the proximal portion can include an opening at its proximal end through which the guidewire can enter the proximal portion, and an opening disposed proximal to light emitting devices 153, where the guidewire then exits the proximal portion.

The length of the linear light source array (i.e., light emitting devices 153 and conductive substrate 155) is only limited by the effective length of expandable member 157. If the linear array is made longer than the expandable member, light emitted from that portion of the linear array will be blocked by blood within the vessel and likely not reach the targeted tissue. As described below in connection with FIGS. 10A-12, the use of a plurality of expandable members enables even longer linear light source arrays (i.e., longer than any single expandable member) to be used in this invention.

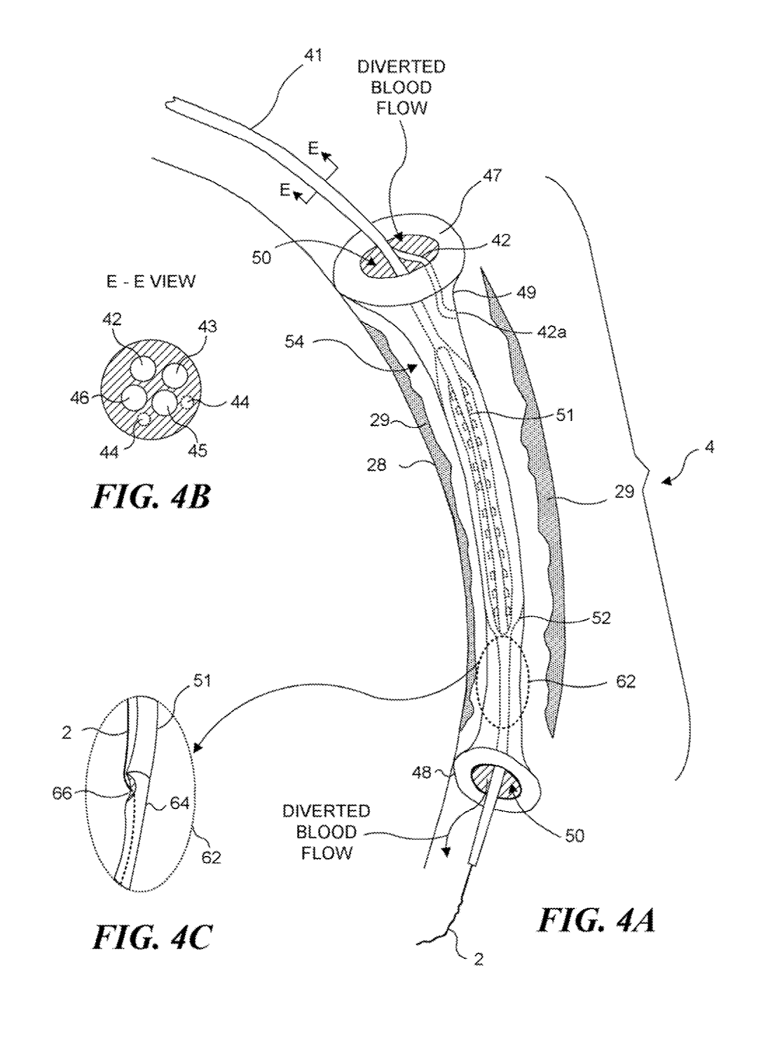

FIG. 4A schematically illustrates another implementation of a light-generating device with an integrated distal protection device, for use during an interventional procedure. Light-generating device 4, shown disposed in saphenous vein graft 28 (which includes treatment areas 29) comprises a multi-lumen catheter 41 having an elongate, flexible body formed from a suitable biocompatible material, such as a polymer or metal. It should be recognized that this catheter may have use beyond the setting of vein graft disease. In particular it may have special utility in the setting of a vessel with one or more branch-points, since it will substantially help to control blood flow from these side branches back into the treatment field in the main vessel, particularly where a long segment is being treated. Thus, the saphenous vein environment is intended to be exemplary, rather than limiting. Catheter 41 includes a proximal torus-shaped protection balloon 47, and a distal torus-shaped protection balloon 48, coupled with an impermeable but light transparent exclusion sleeve 49 that extends between balloon 47 and balloon 48; sleeve 49 thus defines a conduit 50. When catheter 41 is disposed in saphenous vein graft 28 (or in another body lumen) and balloons 47 and 48 are inflated, a portion 54 of saphenous vein graft 28 is defined by the walls of saphenous vein graft 28, sleeve 49, and balloons 47 and 48. Portion 54 is isolated from blood flow, which is diverted around portion 54 through conduit 50, thereby excluding treatment areas 29 (i.e., the lesions) from the vascular lumen, and allowing blood flow to continue during the intervention, which prevents embolization. When inflated, balloons 47 and 48 tend to center the portion of catheter 41 extending between the balloons within the body lumen in which the body lumen catheter 41 is deployed.

Catheter 41 also includes a light source array 51, which is generally consistent with the light source arrays described above. Once again, light source array 51 can be an integral part of catheter 41, or the light source array can be a separate component advanced through a working lumen after catheter 41 is properly positioned, as discussed above. Again, if the light source array is not an integral component of the catheter, then catheter 41 includes a transparent hollow shaft adapted to accommodate the separate light source array, which is introduced into the hollow shaft via a working lumen, also as described above.

Catheter 41 preferably includes an expandable member 52 that is adapted to occlude blood flow through conduit 50 and to perform angioplasty (if desired). Preferably, expandable member 52 encompasses light source array 51 (or the hollow shaft adapted to receive the light source array), and is formed from a suitable transparent biocompatible material, such as, polyurethane, polyethylene, FEP, PTFE or PET. Because expandable member 52 encompasses light source array 51, the expandable member is formed of a light transmissive material, so that light from the light source array can freely pass through the expandable member to reach the target tissue.

As shown in FIG. 4A, light source array 51 and expandable member 52 are disposed within conduit 50, so that sleeve 49 (which defines conduit 50) must also be sufficiently transparent so that light from light source array 51 can freely pass through sleeve 49 to reach target tissue 29. Further, where expandable member 52 is intended to be used to perform angioplasty, sleeve 49 must be sufficiently large and flexible, to accommodate expandable member 52 in its fully expanded state (i.e., when expandable member 52 is inflated to contact the walls of the body lumen in which catheter 41 is disposed). If it is not necessary to perform angioplasty, expandable member 52 is inflated only enough to securely position the light source array within sleeve 49. Preferably, sleeve 49 comprises a polymeric material that transmits light of the wavelength or waveband used for the PDT. Preferably, light source array 51 is rotatable within catheter 41, to enable all portions of the lumen walls to be illuminated. Alternatively, the light source array can include light sources disposed so that light is emitted outwardly from the light source array through substantially a full 360 degrees of arc, to fully illuminate the treatment area.

FIG. 4B illustrates the plurality of lumens included in catheter 41, include a flushing and aspiration lumen 42, an inflation lumen 43, which enables expandable member 52 to be selectively inflated and deflated, optional conductive lumens 44, which accommodate a laser fiber or light emitting diode wire, neither of which are shown, but which can be used in addition to or in place of light source array 51, a balloon inflation lumen 45, which enables balloons 47 and 48 to be selectively inflated and deflated (an additional balloon inflation lumen can be incorporated if it is desired to independently control the inflation/deflation of balloons 47 and 48), and a guidewire lumen 46, which accommodates guidewire 2, to enable catheter 41 to be advanced over the pre-positioned guidewire. Flushing and aspiration lumen 42 is connected to lumen portion 54 through one or more ports 42a that pass through sleeve 49. As described above, the flushing fluid is used to displace blood and debris in portion 54, and to facilitate illumination of target tissue 29 using light source array 51. Exemplary suitable flushing fluids include saline solution, and the other flushing fluids noted above. While a working lumen to accommodate a separate light source array is not specifically shown, it should be understood that such a working lumen is readily included in catheter 41 (such working lumens have been indicated in FIGS. 1D, 2D, and 3D).

To use catheter 41, guidewire 2 is first introduced into the body lumen to be treated and advanced to just beyond the target tissue. Catheter 41 is then advanced into the body lumen over guidewire 2, until light source array 51 (or the hollow shaft adapted to receive the light source array) is disposed adjacent to target tissue 29. Torus-shaped balloons 47 and 48 are then inflated, isolating the portion of the lumen between the balloons. Blood continues to flow through conduit 50. Expandable member 52 is inflated to perform angioplasty (if desired). Saline solution is then flushed and aspirated through flushing and aspiration lumen 42 to maintain a clear light transmission path to the vessel wall essentially free of blood and debris. Expandable member 52 is again inflated, to displace blood flowing within conduit 50, which may interfere with the transmission of light from light source array 51, and to securely position the light source array within sleeve 49. Light source array 51 is energized, preferably for less than about 50 seconds. During the administration of light to the target tissue, expandable member 52 occludes blood flow in conduit 50. It is believed that interrupting blood flow for less than about 50 seconds, followed by enabling blood flow to resume for about 50 seconds (to enable the blood to re-perfuse), should obviate problems that are sometimes encountered when blood flow is occluded for longer intervals in the coronary circulation. Thus, expandable member 52 can be expanded and deflated cyclically, for periods of about 50 seconds each, to administer the desired PDT to a specific target area. Portion 54 (partially defined by balloons 47 and 48) may extend beyond the illumination limits of light source array 51. Preferably, the light source array is then selectively repositioned within portion 54, without having to move balloons 47 and 48, to enable the light source array to administer PDT to all target tissue in portion 54.

One structure that enables light source array 51 to be selectively repositioned without moving balloons 47 and 48 is achieved by forming the catheter body between the balloons from a substantially light transmissive polymer material. Light source array 51 is then slidably disposed in a working lumen in the catheter body, so that the light source array can be repositioned as desired. Such working lumens are shown in FIGS. 1D, 2D and 3D. Expandable member 52 is coupled to the light transmissive portion of the catheter body (i.e., the portion of catheter 41 encompassed by sleeve 49), so that blood flow through sleeve 49 can be occluded when the light source array is energized.

Portion 62 in an enlarged view in FIG. 4C illustrates that catheter 41 preferably includes a hollow tip 64, which is disposed distally of light source array 51 and proximally of balloon 48. Hollow tip 64 includes a side facing orifice 66 that enables catheter 41 to be advanced over guidewire 2 (i.e., light source array 51 does not include a guidewire lumen, and the guidewire is exposed externally to catheter 41, proximate light source array 51). This configuration is shown in greater detail in FIGS. 1A, 2A, 3E, and 3F. Alternatively, but not separately shown, light source array 51 includes a guidewire lumen, or guidewire 2 can be withdrawn once balloons 47 and 48 are inflated, so that a separate light source array can be advanced through the guidewire lumen. Expandable member 52 has been omitted from FIG. 4C, to simplify the Figure.

FIGS. 6, 7A, and 7B are enlarged views of light source arrays that can be used in a light-generating apparatus in accord with the present invention. Light source array 180, shown in FIG. 6, includes a plurality of LEDs 186a and 186b that are coupled to a flexible, conductive substrate 182. LEDs 186a emit light of a first color, having a first wavelength, while LEDs 186b emit light of a different color, having a second wavelength. In an exemplary but not limiting embodiment, the first color is blue and the second color is red. Such a configuration is useful if two different photoreactive agents have been administered, where each different photoreactive agent is activated by light of a different wavelength. A potentially more therapeutically valuable use of two different light sources would be to use two different color light sources (e.g. blue and red) to activate a single photoreactive agent, since this could be used to maximize the generation of singlet oxygen throughout the tissue, including photoreactive agent disposed superficially within the tissue, and photoreactive agent disposed more deeply within the tissue, according to the relative attenuation of the different wavelengths and the relative quantum yields of singlet oxygen at these wavelengths. Light source array 180 optionally includes one or more light sensing elements 184, such as photodiodes or a reference LED, similarly coupled to flexible, conductive substrate 182. Each light sensing element 184 may be coated with a wavelength-specific coating to provide a specific spectral sensitivity, and different light sensing elements can have different wavelength-specific coatings. While light source array 180 is configured linearly, with LEDs on only one side (as the array in light-generating apparatus 150 of FIG. 5), it will be understood that different color LEDs and light sensing elements can be beneficially included in any of the light source arrays described herein.

Because the light source arrays of the present invention are intended to be used in flexible catheters inserted into blood vessels or other body passages, it is important that the light source arrays be relatively flexible, particularly where a light source array extends axially along some portion of the catheter's length. Clearly, the longer the light source array, the more flexible it must be. Light source array 180 (FIG. 6), and the light source array of the light-generating apparatus of FIGS. 1A, 2A, 3B, 4A and 5 are linearly configured arrays that extend axially along a significant distal portion of their respective catheters. A required characteristic of a catheter for insertion into a blood vessel is that the catheter be sufficiently flexible to be inserted into a vessel and advanced along an often tortuous path. Thus, light source arrays that extend axially along a portion of a catheter can unduly inhibit the flexibility of that catheter. FIGS. 7A and 7B schematically illustrate axially extending light source arrays that include strain relief features that enable a more flexible linear array to be achieved.

FIG. 7A shows a linear array 188a having a plurality of light emitting sources 190 (preferably LEDs, although other types of light sources can be employed, as discussed above) mounted to both a first flexible conductive substrate 192a, and a second flexible conductive substrate 192b. Flexible conductive substrate 192b includes a plurality of strain relief features 193. Strain relief features 193 are folds in the flexible conductive substrate that enable a higher degree of flexibility to be achieved. Note that first flexible conductive substrate 192a is not specifically required and can be omitted. Further, strain relief features 193 can also be incorporated into first flexible conductive substrate 192a.

FIG. 7B shows a linear array 188b having a plurality of light emitting sources 190 mounted on a flexible conductive substrate 192c. Note that flexible conductive substrate 192c has a crenellated configuration. As shown, light emitting sources 190 are disposed in each “notch” of the crenellation. That is, light emitting sources 190 are coupled to both an upper face 193a of flexible conductive substrate 192c, and a lower face 193b of flexible conductive substrate 192c. Thus, when light emitting sources 190 are energized, light is emitted generally outwardly away from both upper surface 193a and lower surface 193b. If desired, light emitting sources 190 can be disposed on only upper surface 193a or only on lower surface 193b (i.e., light emitting sources can be disposed in every other “notch”), so that light is emitted generally outwardly away from only one of upper surface 193a and lower surface 193b. The crenellated configuration of flexible conductive substrate 192c enables a higher degree of flexibility to be achieved, because each crenellation acts as a strain relief feature.

External bond wires can increase the cross-sectional size of an LED array, and are prone to breakage when stressed. FIG. 7C schematically illustrates a flip-chip mounting technique that can be used to eliminate the need for external bond wires on LEDs 194 that are mounted on upper and lower surfaces 193c and 193d (respectively) of flexible conductive substrate 19d to produce a light source array 197. Any required electrical connections 195 pass through flexible conductive substrate 192d, as opposed to extending beyond lateral sides of the flexible conductive substrate, which would tend to increase the cross-sectional area of the array. Light source array 197 is shown encapsulated in a polymer layer 123. A guidewire lumen 198a is disposed adjacent to light source array 197. An expandable balloon 199 encompasses the array and guidewire lumen. Note that either, but not both, polymer layer 123 and expandable balloon 199 can be eliminated (i.e., if the expandable balloon is used, it provides protection to the array, but if not, then the polymer layer protects the array).

FIG. 7D shows a linear array 196 including a plurality of light emitting sources (not separately shown) that spirals around a guidewire lumen 198b. Once again, balloon 199 encompasses the guidewire lumen and the array, although if no balloon is desired, a polymer layer can be used instead, as noted above. For each of the implementations described above, the array of light sources may comprise one or more LEDs, organic LEDs, super luminescent diodes, laser diodes, or light emitting polymers ranging from about 1 cm to about 10 cm in length and having a diameter of from about 1 mm to about 2 mm.

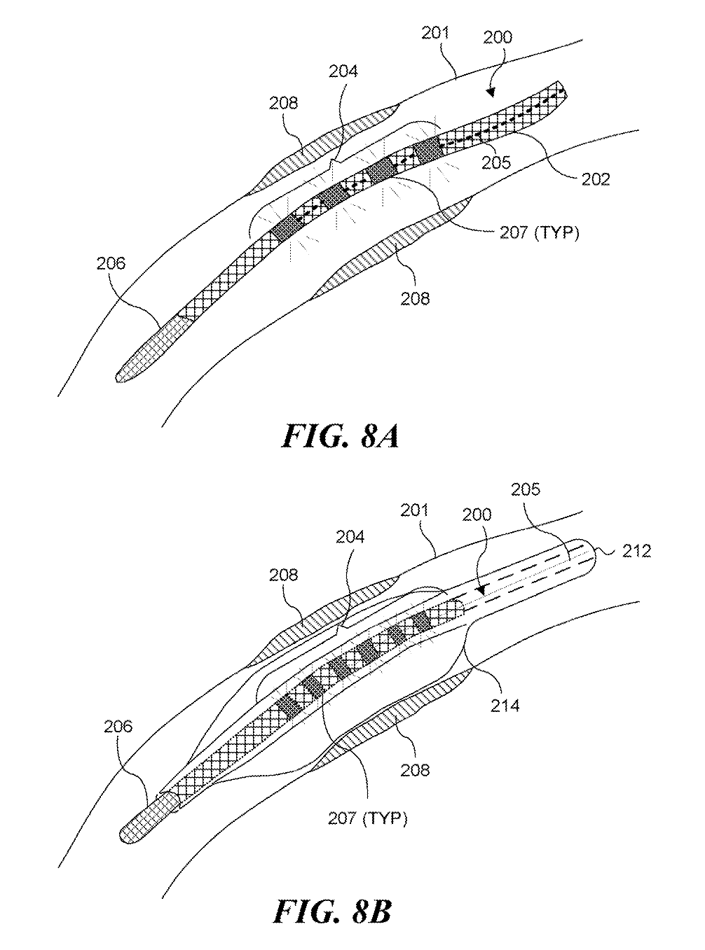

Turning now to FIG. 8A, a light-generating apparatus 200 is shown as the apparatus is being positioned in a blood vessel 201, to administer PDT to treatment areas 208. Light-generating apparatus 200 is simpler in construction than light-generating apparatus of FIGS. 1A, 2A, 3B, 4A, and 5 (each of which is based on a catheter), because light-generating apparatus 200 is based on a guidewire. Light-generating apparatus 200 includes a main body 202, a light source array including a plurality of light sources 207, and an optional spring tip 206. Main body 202 is based on a conventional guidewire, preferably having a diameter ranging from about 0.10 inches to about 0.060 inches. However, main body 202 is distinguishable from a conventional guidewire because main body 202 includes an electrical conductor 205. As will be discussed in greater detail below, either the core can include paired conductors to enable a complete circuit to be achieved, or an additional conductor will be disposed external to the core. Spring tip 206 is also based on a conventional guidewire spring tip. Light source array 204 includes a plurality of light emitting devices 207, each electrically coupled to conductor 205 (alternatively, each light emitting device is coupled to a flexible conductive substrate, that is in turn electrically coupled to conductor 205). While not separately shown, it should be understood that radio-opaque markers can be included at each end of light source array 204, thereby enabling the light source array to be properly positioned relative to treatment areas 208.

In FIG. 8B, light-generating apparatus 200 has been inserted into a balloon catheter 212, and the combination of balloon catheter 212 and light-generating apparatus 200 is shown being positioned in blood vessel 201, also to administer PDT to treatment areas 208. Balloon 214 has been inflated to contact the walls of blood vessel 201, thereby centering the combination of balloon catheter 212 and light-generating apparatus 200 within blood vessel 201 and occluding blood flow that could allow blood to block light emitted from light emitting devices 207 from reaching treatment areas 208. As discussed above, the fluid used to inflate the balloon should readily transmit the wavelengths of light required to activate the photoreactive agent(s) used to treat treatment areas 208. As described above, additives can be added to the fluid to enhance light transmission and diffusion. The fluid will also act as a heat sink to absorb heat generated by light emitting devices 207, and the beneficial effect of the fluid as a coolant can be enhanced by regularly circulating the fluid through the balloon.

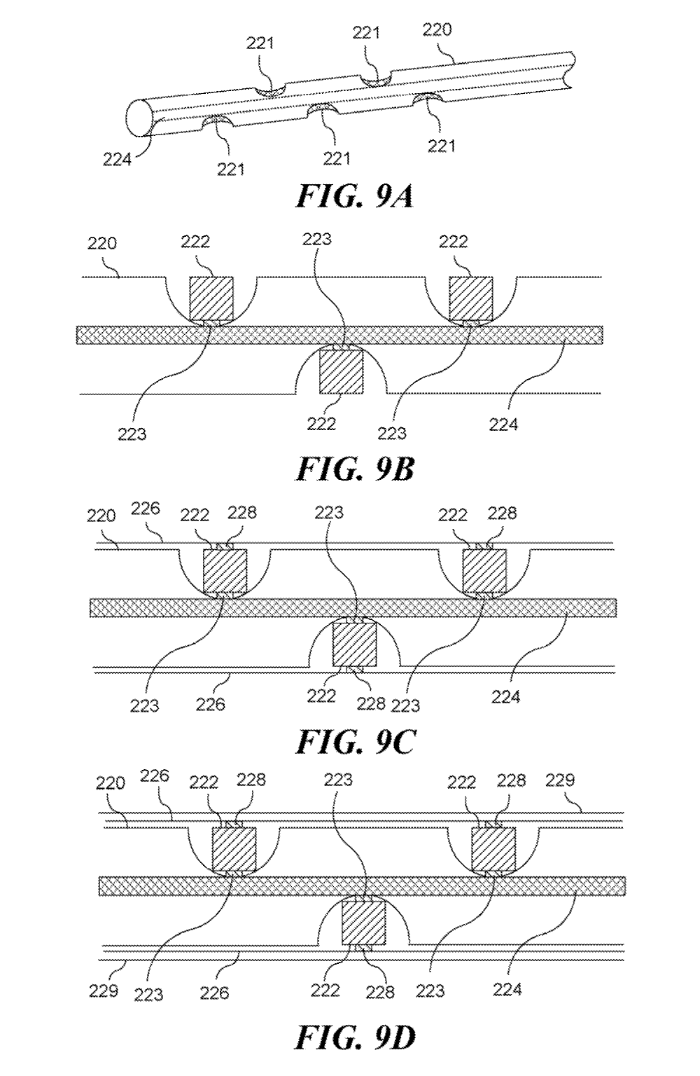

FIGS. 9A-9D provide details showing how light emitting devices can be integrated into guidewires. Referring to FIG. 9A, a solid guidewire 220 includes a conductive core 224 and a plurality of compartments 221 formed in the guidewire around the conductive core. Conductive core 224 is configured to be coupled to a source of electrical energy, so that electrical devices coupled to conductive core 224 can be selectively energized by current supplied by the source. Compartments 221 can be formed as divots, holes, or slots in guidewire 220, using any of a plurality of different processes, including but not limited to, machining, and laser cutting or drilling. Compartments 221 can be varied in size and shape. As illustrated, compartments 221 are arranged linearly, although such a linear configuration is not required. Preferably, each compartment 221 penetrates sufficiently deep into guidewire 220 to enable light emitting devices 222 to be placed into the compartments and be electrically coupled to the conductive core, as indicated in FIG. 9B. A conductive adhesive 223 can be beneficially employed to secure the light emitting devices into the compartments and provide the electrical connection to the conductive core. Of course, conductive adhesive 223 is not required, and any suitable electrical connections can alternatively be employed. Preferably, LEDs are employed for the light emitting devices, although as discussed above, other types of light sources can be used. If desired, only one compartment 221 can be included, although the inclusion of a plurality of compartments will enable a light source array capable of simultaneously illuminating a larger treatment area to be achieved.

Once light emitting devices 222 have been inserted into compartments 221 and electrically coupled to conductive core 224, a second electrical conductor 226, such as a flexible conductive substrate or a flexible conductive wire, is longitudinally positioned along the exterior of guidewire 220, and electrically coupled to each light emitting device 222 using suitable electrical connections 228, such as conductive adhesive 223 as (illustrated in FIG. 9B) or wire bonding (as illustrated in FIG. 9C). Guidewire 220 (and conductor 226) is then coated with an insulating layer 229, to encapsulate and insulate guidewire 220 (and conductor 226). The portion of insulating layer 229 covering light emitting devices 222 must transmit light of the wavelength(s) required to activate the photoreactive agent(s). Other portions of insulating layer 229 can block such light transmission, although it likely will be simpler to employ a homogenous insulating layer that transmits the light. Additives can be included in insulating layer 229 to enhance the distribution of light from the light emitting device, generally as described above.

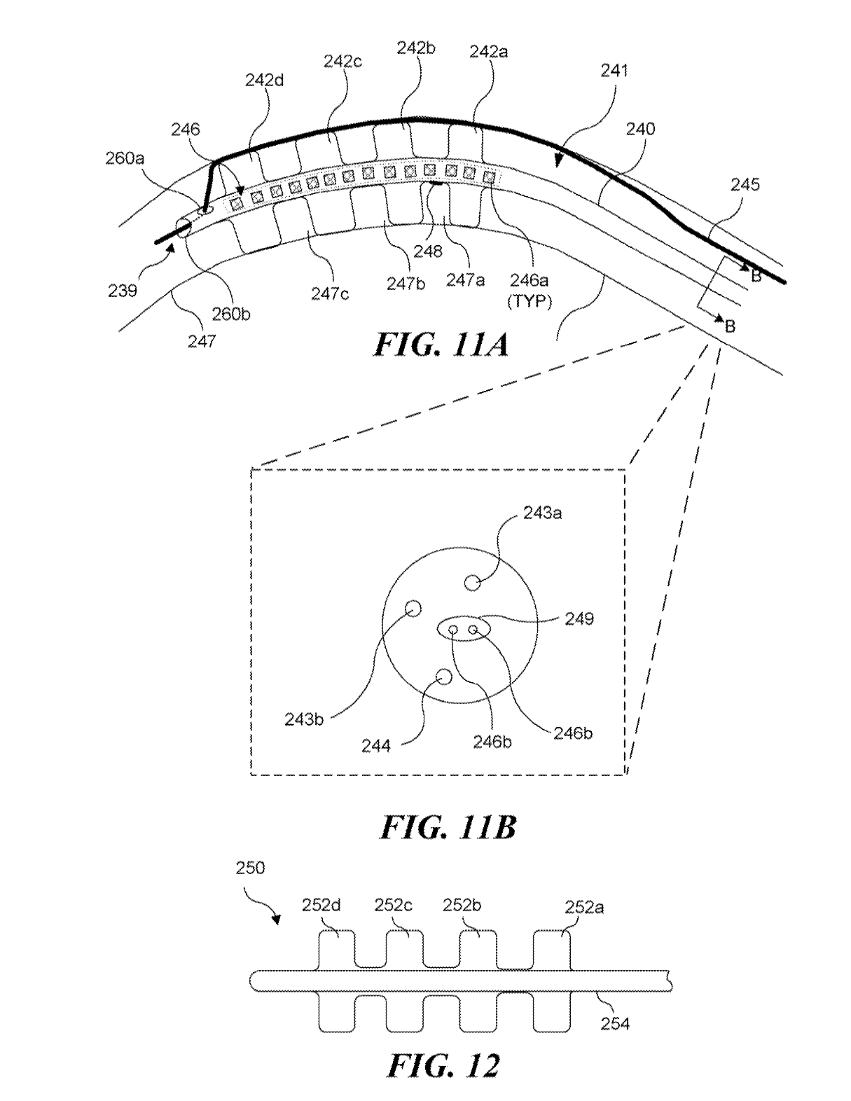

As already noted above, using a plurality of expandable members enables a linear light source array that is longer than any one expandable member to be employed to illuminate a treatment area that is also longer than any one expandable member. FIGS. 10A, 10B, 11A, and 11B illustrate apparatus including such a plurality of expandable members. FIGS. 10A and 10B show an apparatus employed in connection with an illuminated guidewire, while FIGS. 11A and 11B illustrate an apparatus that includes a linear light source array combined with the plurality of expandable members. In each embodiment shown in these Figures, a relatively long light source array (i.e., a light source array having a length greater than a length of any expandable member) is disposed between a most proximally positioned expandable member and a most distally positioned proximal member.

FIG. 10A schematically illustrates a light-generating apparatus 231 for treating relatively long lesions (i.e., lesions of about of 60 mm in length or longer) in a blood vessel 237. Light-generating apparatus 231 is based on a multi-lumen catheter 230 in combination with an illuminated guidewire 235 having integral light emitting devices. Multi-lumen catheter 230 is elongate and flexible, and includes a plurality of expandable members 233a-233d. While four such expandable members are shown, alternatively, more or fewer expandable members can be employed, with at least two expandable members being particularly preferred. As discussed above, such expandable members occlude blood flow and center the catheter in the lumen of the vessel. Multi-lumen catheter 230 and expandable members 233a-233d preferably are formed from a suitable biocompatible polymer, including but not limited to polyurethane, polyethylene, PEP, PTFE, or PET. Each expandable member 233a-233d preferably ranges from about 2 mm to about 10 mm in diameter and from about 1 mm to about 60 mm in length. When inflated, expandable members 233a-233d are pressurized from about 0.01 atmosphere to about 16 atmospheres. With respect to the expandable members discussed in the specification, in each of the various embodiments, the most useful pressures will likely range from about 0.01 to 3 atmospheres. The higher pressures are useful if performing angioplasty; many embodiments consistent with the principles disclosed herein can beneficially employ relatively low-pressure inflation. The lower pressures work with the compliant balloons to occlude flow without causing trauma to the vessel wall. It should be understood that between expandable member 233a and expandable member 233d, multi-lumen catheter 230 is formed of a flexible material that readily transmits light of the wavelengths required to activate the photoreactive agent(s) with which light-generating apparatus 231 will be used. Biocompatible polymers having the required optical characteristics can be beneficially employed. As discussed above, additives such as diffusion agents can be added to the polymer to enhance the transmission or diffusion of light. Of course, all of multi-lumen catheter 230 can be formed of the same material, rather than just the portions between expandable member 233a and expandable member 233d. Preferably, each expandable member 233a-233d is similarly constructed of a material that will transmit light having the required wavelength(s). Further, any fluid used to inflate the expandable members should similarly transmit light having the required wavelength(s).

Referring to the cross-sectional view of FIG. 10B (taken along section lines A-A of FIG. 10A), it will be apparent that multi-lumen catheter 230 includes an inflation lumen 232a in fluid communication with expandable member 233a, a second inflation lumen 232b in fluid communication with expandable members 233b-233c, a flushing lumen 234, and a working lumen 236. If desired, each expandable member can be placed in fluid communication with an individual inflation lumen. Multi-lumen catheter 230 is configured such that flushing lumen 234 is in fluid communication with at least one port 238 (see FIG. 10A) formed through the wall of multi-lumen catheter 230. As illustrated, a single port 238 is disposed between expandable member 233a and expandable member 233b and functions as explained below.

Once multi-lumen catheter 230 is positioned within blood vessel 237 so that a target area is disposed between expandable member 233a and expandable member 233d, inflation lumen 232a is first used to inflate expandable member 233a. Then, the flushing fluid is introduced into blood vessel 237 through port 238. The flushing fluid displaces blood distal to expandable member 233a. After sufficient flushing fluid has displaced the blood flow, inflation lumen 232b is used to inflate expandable members 233b and 233c, thereby trapping the flushing fluid in portions 237a, 237b, and 237c of blood vessel 237. The flushing fluid readily transmits light of the wavelength(s) used in administering PDT, whereas if blood were disposed in portions 237a, 237b, and 237c of blood vessel 237, light transmission would be blocked. An alternative configuration would be to provide an inflation lumen for each expandable member, and a flushing port disposed between each expandable member. The expandable members can then be inflated, and each distal region can be flushed, in a sequential fashion.

A preferred flushing fluid is saline. Other flushing fluids can be used, so long as they are non-toxic and readily transmit light of the required wavelength(s). As discussed above, additives can be included in flushing fluids to enhance light transmission and dispersion relative to the target tissue. Working lumen 236 is sized to accommodate light emitting guidewire 235, which can be fabricated as described above. Multi-lumen catheter 230 can be positioned using a conventional guidewire that does not include light emitting devices. Once multi-lumen catheter 230 is properly positioned and the expandable members are inflated, the conventional guidewire is removed and replaced with a light emitting device, such as an optical fiber coupled to an external source, or a linear array of light emitting devices, such as LEDs coupled to a flexible conductive substrate. While not specifically shown, it will be understood that radio-opaque markers such as those discussed above can be beneficially incorporated into light-generating apparatus 231 to enable expandable members 233a and 233d to be properly positioned relative to the target tissue.

Still another embodiment of the present invention is light-generating apparatus 241, which is shown in FIG. 11A disposed in a blood vessel 247. Light-generating apparatus 241 is similar to light-generating apparatus 231 describe above, and further includes openings for using an external guide wire, as described above in connection with FIG. 5. An additional difference between this embodiment and light-generating apparatus 231 is that where light emitting devices were not incorporated into multi-lumen catheter 230 of light-generating apparatus 231, a light emitting array 246 is incorporated into the catheter portion of light-generating apparatus 241.

Light-generating apparatus 241 is based on an elongate and flexible multi-lumen catheter 240 that includes light emitting array 246 (including a plurality of light sources 246a) and a plurality of expandable members 242a-242d. Light emitting array 246 preferably comprises a linear array of LEDs. As noted above, while four expandable members are shown, more or fewer expandable members can be employed, with at least two expandable members being particularly preferred. The materials and sizes of expandable members 242a-242d are preferably consistent with those described above in conjunction with multi-lumen catheter 230. The walls of multi-lumen catheter 240 proximate to light emitting array 246 are formed of a flexible material that does not substantially reduce the transmission of light of the wavelengths required to activate the photoreactive agent(s) with which light-generating apparatus 241 will be used. As indicated above, biocompatible polymers having the required optical characteristics can be beneficially employed, and appropriate additives can be used. Preferably, each expandable member is constructed of a material and inflated using a fluid that readily transmit light of the required wavelength(s).

Referring to the cross-sectional view of FIG. 11B (taken along section line B-B of FIG. 11A), it can be seen that multi-lumen catheter 240 includes an inflation lumen 243a in fluid communication with expandable member 242a, a second inflation lumen 243b in fluid communication with expandable members 242b-c, a flushing lumen 244, and a working lumen 249. Again, if desired, each expandable member can be placed in fluid communication with an individual inflation lumen. Multi-lumen catheter 240 is configured so that flushing lumen 244 is in fluid communication with a port 248 (see FIG. 11A) formed in the wall of multi-lumen catheter 240, which enables a flushing fluid to be introduced into portions 247a-247c of blood vessel 247 (i.e., into those portions distal of expandable member 242a). Those portions are isolated using inflation lumen 243b to inflate expandable members 242b-242d. The flushing fluid is selected as described above. Working lumen 249 is sized to accommodate light emitting array 246. Electrical leads 246b within working lumen 249 are configured to couple to an external power supply, thereby enabling the light source array to be selectively energized with an electrical current. A distal end 239 of multi-lumen catheter 240 includes an opening 260a in the catheter side wall configured to enable guidewire 245 (disposed outside of multi-lumen catheter 240) to enter a lumen (not shown) in the distal end of the catheter that extends between opening 260a and an opening 260b, thereby enabling multi-lumen catheter 240 to be advanced over guidewire 245.

FIG. 12 shows an alternative embodiment of the light-generating apparatus illustrated in FIGS. 10A, 10B, 11A, and 11B. A light-generating apparatus 250 in FIG. 12 is based on a multi-lumen catheter having an elongate, flexible body 254 formed from a suitable biocompatible polymer and expandable members 252a-252d. As indicated above, at least two expandable members are particularly preferred. The difference between light-generating apparatus 250 and light-generating apparatus 231 and 241, which were discussed above, is that the expandable members in light-generating apparatus 250 are fabricated as integral portions of body 254, while the expandable members of light-generating apparatus 231 and 241 are preferably implemented as separate elements attached to a separate catheter body.

It should be recognized that in any of the embodiments disclosed above wherein a first expandable member is disposed proximal of the light source or light source array, and a second expandable member is disposed distal of the light source/array, activating the first and second member will isolate a region of the vessel proximate the light source/array. The photoreactive drug can then be administered into the region between the first expandable member and the second expandable member, thereby increasing photoreactive drug uptake in tissue surrounding the region, while limiting introduction of the photoreactive drug to other portions of a patient's body. This aspect of the invention can be implemented even where one of the expandable members only partially occludes blood flow (i.e., where that expandable member is primarily an anchoring member, as opposed to an occlusion member), so long as the blood flow naturally moves from the anchoring member to the other expandable member.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

1. Apparatus for illuminating a portion of a body lumen, comprising:
(a) an elongate flexible body having a proximal end, a distal end, and a plurality of lumens, said plurality of lumens including a guide wire lumen that extends through the elongate flexible body, but not beyond an opening in the distal end of the guide wire lumen, which is at the distal end of the elongate flexible body;
(b) a light emitting portion that extends distally beyond the distal end of the elongate flexible body, the light emitting portion being fabricated of a material that transmits light to illuminate tissue disposed adjacent to the light emitting portion, the light emitting portion being positioned relative to the elongate flexible body such that the light emitting portion does not block the opening for the guide wire lumen in the elongate flexible body, the light emitting portion being fixedly coupled to the distal end of the elongate flexible body; and
(c) a tip disposed at a distal end of the light emitting portion, the tip having an internal passage that connects a first orifice disposed on one side of the tip, with a second orifice disposed on a distal face of the tip, to enable a guide wire extending distally from the opening at the distal end of the guide wire lumen and passing externally of the light emitting portion, to enter the first opening, following the passage through the tip, and passing out through the second opening, so that the elongate flexible body, the light emitting portion, and the tip can be advanced over the guide wire to a desired position in the body lumen.

2. The apparatus of claim 1, wherein the light emitting portion has a smaller diameter than the elongate flexible body.

3. The apparatus of claim 1, further comprising an occluding member disposed proximal of the light emitting portion, the occluding member being inflatable to expand outwardly toward an inner surface of a body lumen in which the apparatus is disposed, the occluding member including a plurality of lobes that when inflated only partially occlude a bodily fluid flow through the body lumen.

4. The apparatus of claim 1, wherein the light emitting portion includes a flexible substrate having crenellations comprising notches formed on alternating opposite sides of the substrate, and having a plurality of discrete light emitting sources mounted in at least some of the notches, the crenellations providing an enhanced flexibility for the substrate, thereby enabling the light emitting portion to more readily navigate tortuous body lumens.

5. The apparatus of claim 1, further comprising an integral source of light disposed within the light emitting portion, which when energized, emits light in one or more desired wavelengths or wavebands, the integral source of light comprising an array of discrete light sources that are distributed longitudinally within the light emitting portion, and wherein the plurality of lumens in the elongate flexible body includes a power lumen through which a power lead extends, a proximal end of the power lead being connectable to a power source to provide an electrical current to energize the array of discrete light sources.

6. The apparatus of claim 1, wherein the plurality of lumens in the elongate flexible body includes a working lumen, and wherein the light emitting portion is hollow and is coupled to the working lumen to receive a source of light that has been advanced through the working lumen and into the light emitting portion, the source of light emitting light in one or more desired wavelengths or wavebands when energized.

7. The apparatus of claim 1, further comprising a distal protection device that is attached to the apparatus distal of the light emitting portion, the distal protection device being selectively changeable between a first shape and a second shape, so that when in the first shape, the distal protection device generally conforms to an outer surface of a portion of the apparatus to which the distal protection device is attached, and when changed to have the second shape, the distal protection device extends outwardly from the portion of the apparatus to an inner surface of a body lumen in which the distal protection device is disposed, enabling the distal protection device to either at least partially occlude a flow of a bodily fluid through the body lumen, or filter the flow of the bodily fluid through the body lumen, to prevent debris from moving past the distal protection device.

8. The apparatus of claim 7, wherein the distal protection device comprises an inflatable member that is inflated with a pressurized fluid to change from the first shape to the second shape.

9. The apparatus of claim 7, wherein the distal protection device is formed of a shape memory material that changes between the first shape and the second shape in response to a change in temperature.

10. The apparatus of claim 9, wherein heat associated with the light emitted from the light emitting portion causes a change in the temperature of the distal protection device, so that the distal protection device changes between the first shape and the second shape.

11. The apparatus of claim 9, wherein the plurality of lumens includes a power lumen, further comprising a heating element that is disposed adjacent to the distal protection device, the heating element being selectively energized with an electrical current supplied through a lead that extends through the power lumen, heat from the heating element produced when it is energized changing the temperature of the distal protection device, so that the distal protection device changes from the first shape to the second shape.

12. The apparatus of claim 7, further comprising an occluding member disposed proximal of the light emitting portion, the occluding member being inflatable to expand outwardly toward an inner surface of a body lumen in which the apparatus is disposed, and when thus inflated, at least partially occluding bodily fluid flow through the body lumen, so that when the distal protection device changes to the second shape, the occluding member and the distal protection member cooperate to generally isolate a section of the body lumen in which the light emitting portion is disposed, and wherein the plurality of lumens includes a flushing lumen through which a biocompatible flushing fluid can be conveyed into the section of the body lumen that is isolated, the flushing fluid displacing a bodily fluid that was in the section and being characterized by its capability for transmitting light emitted from the light emitting portion, to adjacent tissue.

13. The apparatus of claim 1, further comprising a plurality of expandable members that are light transmissive and are disposed around and longitudinally spaced-apart along the light emitting portion, the plurality of lumens including at least one inflation lumen for conveying a pressurized fluid to expand the plurality of expandable members outwardly into contact with an inner surface of a body lumen in which the apparatus is disposed, isolating spaced-apart segments of the body lumen.

14. The apparatus of claim 13, wherein the plurality of lumens include a flushing lumen for conveying a biocompatible flushing fluid into the spaced-apart segments of the body lumen, and an aspiration lumen for conveying the flushing fluid and any debris proximally from the spaced-apart segments, if the flushing lumen is not instead employed for doing so.

15. The apparatus of claim 1, wherein a distal end portion of the guide wire includes a plurality of light emitting sources that emit light having at least one desired wavelength or waveband.

16. Apparatus for illuminating a portion of a body lumen, comprising:
(a) an elongate flexible body having a proximal end, a distal end, and a plurality of lumens, including a guide wire lumen that terminates at an opening disposed at the distal end of the elongate flexible body;
(b) a light emitting portion being fixedly coupled to the distal end of the elongate flexible body and extending distally beyond the distal end of the elongate flexible body, such that the opening in the distal end of the elongate flexible body for the guide wire lumen is not obstructed by the light emitting portion, the light emitting portion including either:
(i) an integral source of light configured to emit light having a desired characteristic emission wavelength or waveband, or
(ii) a light emitting lumen coupled to a working lumen in the elongate flexible body, to receive an elongate flexible source of light that is advanced through the working lumen and into the light emitting lumen of the light emitting portion, the elongate flexible source of light emitting light having the desired characteristic emission wavelength or waveband; and
(c) a tip disposed distal of the light emitting portion, the tip having a distal face including a first orifice, and a side surface including a second orifice, the first and second orifices opening into an interior of the tip.

17. The apparatus of claim 16, further comprising a guide wire that passes through the guide wire lumen, out through the opening of the guide wire lumen in the elongate flexible body, continues externally past the light emitting portion and into the second orifice of the tip, through the interior of the tip, and out from the first orifice, the guide wire enabling the elongate flexible body, the light emitting portion, and the tip to be advanced through a body lumen to a desired site where the light emitted from the light emitting portion can be administered to tissue on an inner surface of the body lumen

18. The apparatus of claim 16, wherein the light emitting portion has a smaller diameter than the elongate flexible body.

19. The apparatus of claim 16, wherein the integral source of light in the light emitting portion includes a flexible substrate having crenellations comprising notches formed on alternating opposite sides of the substrate, and having a plurality of discrete light emitting sources mounted in at least some of the notches, the crenellations providing an enhanced flexibility for the substrate, thereby enabling the light emitting portion to more readily navigate tortuous body lumens.

20. The apparatus of claim 16, wherein the elongate flexible source of light that is advanced through the working lumen and into the light emitting lumen of the light emitting portion includes a flexible substrate having crenellations comprising notches formed on alternating opposite sides of the substrate, and having a plurality of discrete light emitting sources mounted in at least some of the notches, the crenellations providing an enhanced flexibility for the substrate, thereby enabling the elongate flexible source of light to more readily navigate tortuous body lumens

21. The apparatus of claim 16, further comprising an occluding member disposed proximal of the light emitting portion, the occluding member being inflatable to expand outwardly toward an inner surface of a body lumen in which the apparatus is disposed, the occluding member including a plurality of lobes that when inflated only partially occlude a bodily fluid flow through the body lumen.

22. The apparatus of claim 16, further comprising a distal protection device that is attached to the apparatus distal of the light emitting portion, the distal protection device being selectively changeable between a first shape and a second shape, so that when in the first shape, the distal protection device generally conforms to an outer surface of a portion of the apparatus to which the distal protection device is attached, and when changed to have the second shape, the distal protection device extends outwardly from the portion of the apparatus to an inner wall of a body lumen in which the distal protection device is disposed, enabling the distal protection device to either at least partially occlude a flow of a bodily fluid through the body lumen, or filter the flow of the bodily fluid through the body lumen, to prevent debris from moving past the distal protection device.

23. The apparatus of claim 16, wherein the plurality of lumens includes a flushing lumen for conveying a biocompatible flushing fluid into a region adjacent to the light emitting portion, the flushing fluid displacing a bodily fluid in the region, the flushing lumen also being used for aspirating the flushing fluid and debris from the region, unless a separate aspiration lumen is also included in the plurality of lumens.

24. A method for administering light to tissue at a desired site within a body lumen, comprising the steps of:
(a) providing a light administering apparatus that includes:
(i) an elongate flexible body, the elongate flexible body having a proximal end, a distal end, and a plurality of lumens, the plurality of lumens including a guide wire lumen that terminates at an opening disposed at a distal end of the elongate flexible body;
(ii) a light emitting portion fixedly attached to the distal end of the elongate flexible body and extending distally thereof; and
(iii) a tip disposed distally of the light emitting portion and having a first orifice disposed in a distal face of the tip, and a second orifice disposed in a side of the tip, the first and second orifices being coupled through the interior of the tip;
(b) threading a guide wire through the light administering apparatus, where the guide wire passes in sequence:
(i) through the guide wire lumen;
(ii) externally past the light emitting portion;
(iii) into the second orifice;
(iv) through the interior of the tip; and
(v) out through the first orifice;
(c) advancing a distal end of the guide wire at least just past the desired site within the body lumen;
(d) advancing the light administering apparatus over the guide wire until the light emitting portion is disposed at the desired site in the body lumen;
(e) displacing a bodily fluid from a region within the body lumen that encompasses the light emitting portion using a biocompatible fluid that is conveyed into the region through the elongate flexible body; and
(f) energizing a source of light disposed within the light emitting portion so that the source of light emits light of at least one characteristic wavelength or waveband to illuminate the tissue at the desired site within the body lumen.

25. The method of claim 24, wherein the step of displacing the bodily fluid from the region comprises the steps of:
(a) generally occluding a flow of the bodily fluid past the light emission portion by expanding at least one occluding member disposed proximal of the light emission portion, outwardly into contact with an inner surface of the body lumen; and
(b) conveying the biocompatible flushing fluid through a flushing lumen in the elongate flexible body and into the region to displace the bodily fluid in the region.

26. The method of claim 24, further comprising the step of selectively deploying a distal protection device that is attached to a portion of the light administering apparatus distal of the light emitting portion, so that the distal protection device extends outwardly from the portion of the light administration apparatus and into contact with an inner surface of the body lumen, enabling the distal protection device to either at least partially occlude a flow of a bodily fluid through the body lumen, or filter the flow of the bodily fluid through the body lumen, to prevent debris from moving past the distal protection device.

27. The method of claim 26, wherein the distal protection device is formed of a shape memory material, and wherein the step of selectively deploying the distal protection device comprises the step of selectively heating the shape memory material of the distal protection device so that the distal protection device changes shape to a deployed state in accord with shape memory characteristics of the shape memory material.

28. The method of claim 24, wherein the step of displacing the bodily fluid from the region comprises the steps of:
(a) providing an expandable member that generally encompasses the light emitting portion, the expandable member being formed of a material that readily transmits the light emitted by the light emitting portion; and
(b) using a pressurized fluid supplied through one of the plurality of lumens in the elongate flexible body, expanding the expandable member outwardly sufficiently so that the expandable member contacts an inner surface of the body lumen, to generally center the light emitting portion within the body lumen, the pressurized fluid modifying light transmission characteristics within a volume encompassed by the expandable member in a desirable manner, while excluding a bodily fluid from the volume.

29. The method of claim 28, further comprising the step of at least periodically circulating the pressurized fluid through the volume to provide cooling of the light emitting portion.
